(19) United States
(12) Patent Application Publication
(10) Pub. No.: US 2026/0177561 A1
(43) Pub. Date: Jun. 25, 2026

(54) BIOMARKERS FOR THE DIAGNOSIS OF DISEASES OR DISORDERS OF THE FEMALE REPRODUCTIVE TRACT

(71) Applicants: Hera Biotech, Inc., San Antonio, TX (US); Universität Bern, Bern (CH)

(72) Inventors: Michael D. Mueller, Bern (CH); Lea Duempelmann, Bern (CH); Thomas Andrieu, Bern (CH); Peter Nestorov, Basel (CH); Cinzia Donato, Basel (CH); Angelo Duo, Basel (CH); Albulena Toska, Basel (CH)

(21) Appl. No.: 19/124,085

(22) PCT Filed: Oct. 26, 2022
(86) PCT No.: PCT/EP2022/080025

§ 371 Date: Apr. 24, 2025

(51) Int. Cl. G01N 33/68; A61K 9/00

(52) U.S. Cl. CPC G01N 33/6896; A61K 9/0019; G01N 33/6893; A61K 2300/00

(57) ABSTRACT

The invention relates to methods for diagnosing, predicting disease development, disease progression and/or disease outcome, predicting susceptibility to treatment, and/or classification in the context of diseases or disorders of the female reproductive tract, in particular endometriosis, wherein biomarkers of Table 1 are determined, e.g. CCL5 and/or NEAT1. The invention further relates to pharmaceutical products for use in patients stratified according to the methods of the invention and to compositions comprising reagents for the detection of the biomarkers of Table 1 for the diagnosis of diseases or disorders of the female reproductive tract.

The invention relates to methods for diagnosing, predicting disease development, disease progression and/or disease outcome, predicting susceptibility to treatment and/or classification in the context of diseases or disorders of the female reproductive tract, wherein biomarkers of Table 1 are determined. The invention further relates to pharmaceutical products for use in patients stratified according to the methods of the invention and to compositions comprising reagents for the detection of the biomarkers of Table 1 for the diagnosis of diseases or disorders of the female reproductive tract.

Diseases or disorders of the female reproductive tract can occur as a result of disease in one of the reproductive organs. These disorders often present as altered menstruation, pelvic pain, or infertility during the reproductive years. Diseases or disorders of the female reproductive tract include without limitation endometrial cancer, ovarian cancer, adenomyosis and endometriosis.

Ovarian cancer is a group of diseases that originates in the ovaries, or in the related areas of the fallopian tubes and the peritoneum. Ovarian cancer is often diagnosed in later stages of the disease (stage III and IV, metastatic) due to its asymptomatic onset. Early detection of ovarian cancer implicates a better response to treatments. Ovarian cancer is relatively rare (0.0146 prevalence in women), however BRCA1 and BRCA2 mutations, and lynch syndrome are high risk factors for ovarian cancer. Ovarian cancer is divided into several subtypes: Invasive epithelial, stromal, germ cell-tumor, fallopian-tumor. 5-years survival rate depends on the subtype and it ranges around 95% for localized tumors, between 50 and 94% in regional tumors and 30-70% in distant settings.

Diagnosis of ovarian cancer comprises several imaging techniques such as MRI and CT-scans as well as blood tests.

Endometriosis is an estrogen dependent disease characterized by the growth of endometrial tissue outside the uterus. These “lesions” can be found throughout the peritoneal cavity, which allows separating largely the disease into three distinct subtypes: superficial peritoneal (SUP), ovarian (OMA), and deeply infiltrating endometriosis (DIE) (Chapron, C., et al., Hum Reprod, 2011. 26(8): p. 2028-35). DIE is considered the most severe form of the disease and defined by lesions that infiltrate more than 5 cm into the underlying tissue. Endometriosis is extremely prevalent, occurring in up to 10% of reproductive aged women but also extremely heterogeneous since it can manifest in a variety of symptoms and medical complications. It can lead to significant pelvic pain, subfertility, pregnancy complications (Giudice, L. C., Clinical practice. Endometriosis. N Engl J Med, 2010. 362(25): p. 2389-98) and has been associated with an increased chance of developing ovarian cancer later in life (Wentzensen, N., et al., J Clin Oncol, 2016. 34(24): p. 2888-98).

The pathogenesis of endometriosis is still unclear. The most commonly accepted theory is the one by Sampson of retrograde menstruation originally proposed in 1927 (Sampson, J. A., Am J Pathol, 1927. 3(2): p. 93-110 43). Under this theory, viable endometrial epithelial and stromal cells are refluxed back through the fallopian tubes into the peritoneal cavity during menstruation. These cells are able to avoid immune surveillance and ultimately attach and grow on the mesothelial cell lining. However, up to 90% of women experience retrograde menstruation but only some of these develop lesions, thus other aberrant biological factors must be involved.

At present, there are no non-invasive diagnostic tests, and direct observation by laparoscopy is the gold standard for diagnosis. If endometriosis is detected, during a laparoscopy, these lesions are removed, and part of this tissue is examined to confirm the diagnosis. In addition, the peritoneal fluid which would inform about the lesion microenvironment is routinely removed. Influenced by menstrual hormones, both disease progression and the accompanying symptoms are cyclically stimulated.

Growth of the lesions can be controlled by hormonal modulation, unfortunately, this represents an inadequate option for women wishing to retain fertility. Moreover, resistance to hormonal therapy has been recently observed. The prevalence and need for surgery put an extraordinary strain on health care systems and economic productivity.

Thus, there is a need for improved methods to examine and/or predict disease or disorder of the female reproductive tract-related parameters such as diagnosis, disease development, disease progression, disease outcome and/or susceptibility to treatment.

The above technical problem is solved by the embodiments disclosed herein and as defined in the claims.

Accordingly, the invention relates to, inter alia, the following embodiments:
1. A method for determining an endometriosis state in an endometrial tissue sample of a female subject, the method comprising the steps of:
   i) determining RNA levels of at least two biomarkers in an endometrial tissue sample of a female subject, wherein the biomarkers comprise or consist of:
      a) CCL5 and/or NEAT1; and/or
      b) further biomarker(s) selected from Table 1; and
   ii) determining an endometriosis status in the endometrial tissue sample based on the RNA levels of the at least two biomarkers of i).
2. The method of embodiment 1, wherein
   a) i) an alteration compared to a reference value of one or more biomarkers selected from Table 2 is indicative of an endometriosis disease state; and/or
      ii) an alteration compared to a reference value of NEAT1 and/or (a) further biomarker(s) selected from Table 3 is indicative of an non-endometriosis disease state; and
   b) wherein the reference value is indicative of a healthy status.
3. A method for determining an endometriosis state based on a plurality of endometrial cells, the method comprising the steps of:
   i) determining a frequency of cells expressing RNA of at least two biomarkers in a plurality of cells of an endometrial sample of a female subject, wherein the biomarkers comprise or consist of:
      a) CCL5 and/or NEAT1; and/or
      b) further biomarker(s) selected from Table 1; and
   ii) determining an endometriosis state based on the frequency determined in i).
4. The method of embodiment 3, wherein
   a) i) an altered frequency of cells expressing one or more biomarkers selected from Table 2 compared to a reference frequency is indicative of an endometriosis disease state; and/or
      ii) an altered frequency of cells expressing NEAT1 and/or (a) further biomarker(s) selected from Table 3 compared to a reference frequency is indicative of a non-endometriosis disease state; and
   b) wherein the reference value is indicative of a healthy status.
5. The method of any one of embodiments 1 to 4, wherein the method comprises at least one step of pre-selecting cells
   i) selected from the group consisting of:
      a) immune cells selected from the group consisting of B-cells, T-cells, dendritic cells and macrophages;
      b) epithelial cells selected from the group consisting of basal cells, ciliated cells and unciliated cells;
      c) endothelial cells; and
      d) smooth muscle cells; and/or
   ii) with at least one cell lineage marker, preferably using at least one cell lineage marker selected from the group of:
      a) CD14, CD16, CD45, CD15, CD11b;
      b) EpCAM and KRT18; and
      c) COL18A1, COL4A2, COL4A1, VIM or CALD1.
6. The method of any one of embodiments 1 to 5, wherein at least 3, 4, 5, 6, 7, 8 or 9 biomarkers are determined.
7. The method of any one of embodiments 1 to 6, wherein the method additionally comprises determining or retrieving at least one non-molecular marker, preferably wherein the non-molecular marker comprises a marker selected from the group consisting of: age, weight, BMI, gravidity, parity, ethnicity, fertility status, previous laparoscopies, previous use of medication and other gynaecological disorders.
8. The method of any one of embodiments 1 to 7, wherein the method is at least partially computer-implemented and wherein the RNA levels are determined by retrieving data indicative for the RNA levels.
9. The method of any one of embodiments 1 to 8, wherein the sample is a proliferative phase sample or wherein the cells are cells obtained during the proliferative phase.
10. The method of embodiment 9, wherein an increase of one or more biomarkers selected from Table 4 is indicative of an endometriosis disease state and/or wherein an increase of one or more biomarkers selected from Table 5 is indicative of a non-endometriosis disease state.
11. A method for determining the validity of an endometriosis state, the method comprising the steps of:
i) determining an endometriosis state according to embodiment 1 to 8;
ii) determining or retrieving the menstrual cycle status of the subject at the timepoint of obtainment of the endometrial cells or of the endometrial sample;
iii) determining the validity of the endometriosis state, based on the menstrual cycle status, preferably wherein the validity is considered higher if the menstrual cycle status is the proliferative phase than if the menstrual cycle status is in a different menstrual cycle state.
12. The method of embodiment 11, wherein determining the menstrual cycle status comprises determining the RNA level of at least one menstrual cycle biomarker.
13. The method of embodiment 12, wherein the menstrual cycle marker comprises a marker from Table 8.
14. A method for prediction of disease outcome, disease development and/or disease progression of a female subject having endometriosis or at risk of having a endometriosis, the method comprising the steps of:
   a) determining an endometriosis state according to the method of any one of embodiments 1 to 10;
   b) comparing the endometriosis state determined in a) to a prediction reference pattern; and
   c) predicting disease outcome disease development and/or disease progression of the female subject based on the comparison in step b).
15. The method of embodiment 13, wherein
1.) determining an increased frequency of cells expressing the biomarker(s) from Table 2 compared to the prediction reference pattern; and/or
2.) determining an increased level of the biomarker(s) from Table 2 compared to the reference pattern
   is indicative for worsening of disease outcome more likely disease development and/or more likely disease progression.
16. The method of embodiment 14 or 15, wherein
   1.) determining an increased frequency of cells expressing the biomarker(s) from Table 3 compared to the prediction reference pattern; and/or
   2.) determining an increased level of NEAT1 and/or (a) further biomarker(s) from Table 3 compared to the reference pattern
      is indicative for improvement of disease outcome less likely disease development and/or less likely disease progression.
17. A method for predicting susceptibility to a treatment for endometriosis of a female subject having endometriosis or at risk of having endometriosis, the method comprising the steps of:
   a) a) determining an endometriosis state according to the method of any one of embodiments 1 to 10;
   b) comparing the endometriosis state to a susceptibility reference pattern; and
   c) predicting susceptibility to a treatment for endometriosis of the female subject based on the comparison in step b).
18. The method of embodiment 17 wherein the treatment for endometriosis is a treatment selected from the group consisting of: pain medication, hormonal therapy, fertility treatment and surgery.
19. The method of any one of embodiments 14 to 18, wherein the susceptibility reference pattern or the prediction reference pattern is obtained from reference subjects, wherein at least one of the reference subjects has been diagnosed with endometriosis.
20. The method of embodiment 19, wherein obtaining the susceptibility reference pattern or the prediction reference pattern from reference subjects comprises a machine-learning technique, preferably a convolutional neural network and/or logistic regression.
21. A method for classification of a female subject having a endometriosis or at risk of having a endometriosis into a class, the method comprising the steps of:
a. i) determining an endometriosis state according to the method of any one of embodiments 1 to 10;
   ii) predicting a disease outcome, disease development and/or disease progression of a female subject according to the method of any one of embodiments 14 to 16, 19 or 20; and/or
   iii) predicting susceptibility to a treatment for endometriosis of a female subject according to the method of any one of embodiments 17 to 20; and
b. classifying the female subject according to the frequency determined in i), signature determined in ii), prediction of iii), and/or prediction in iv).
22. The method of embodiment 21, wherein at least one class is indicative of the stage and/or severity of the endometriosis.
23. A composition comprising reagents for the detection of biomarkers for the diagnosis of endometriosis, the biomarkers comprising or consisting of at least two markers from Table 1.
24. A pharmaceutical product comprising a compound against endometriosis for use in treatment of a female subject that is predicted as susceptible to a treatment for endometriosis according to the method of embodiment 17 to 20.
25. The pharmaceutical product of embodiment 24, wherein the compound against endometriosis is selected from the group of: ibuprofen, naproxen, oxycodone, desogestrel, dienogest, levonorgestrel, clomiphene citrate, gonadotropins, metformin, letrozole and bromocriptine.
26. The method of any one of embodiments 1 to 22, the composition of embodiment 23 or the pharmaceutical product of embodiment 24 or 25, wherein the endometriosis is selected from the group of: peritoneal endometriosis, endometriomas, deeply infiltrating endometriosis, tubal endometriosis and abdominal wall endometriosis.
27. The method of any one of embodiments 1 to 22, 26, the composition of embodiment 23 or 26 or the pharmaceutical product of any one of embodiments 24, 25 or 26, wherein the endometriosis at rASRM II, III, or IV stage.
28. A computer program product comprising instructions to execute the method of any one of embodiments 8 to 22, 26, 27, wherein the method is computer-implemented.

Accordingly, in a first embodiment, the invention relates to a method for determining an endometriosis state in an endometrial tissue sample of a female subject, the method comprising the steps of: i) determining RNA levels of at least two biomarkers in an endometrial tissue sample of a female subject, wherein the biomarkers are selected from Table 1; and ii) determining an endometriosis status in the endometrial tissue sample based on the RNA levels of the at least two biomarkers of i).

The term “endometriosis state”, as used herein, refers to measure or pattern indicative of whether or not a subject has endometriosis based on an endometrial RNA expression pattern. The endometriosis state may further indicate what kind of endometriosis the sample is indicative of. In some embodiments, the endometriosis state can further indicate treatment susceptibility, disease progression and/or disease outcome if compared to the respective reference.

The “RNA level” described herein can be any RNA level that is indicative of the biomarkers described herein, preferably an mRNA level of the respective biomarker described herein.

The term “endometrial tissue sample” refers to any sample that is obtained by from the endometrium. The sample can be any sample from the endometrium that comprises a sufficient amount of RNA for analysis. Typically, an instrument is passed through the cervix into the uterus to collect the tissue sample. In some embodiments, the endometrial tissue sample described herein is a biopsy of the endometrium. In other embodiments, the endometrial tissue sample described herein is obtained through a swab. In other embodiments, the endometrial tissue sample described herein is obtained from menstrual blood.

In certain embodiments, the invention relates to a method for determining the frequency of disease or disorder of the female reproductive tract-signature cells in a plurality of cells, the method comprising the steps of: i) determining the levels of expression of at least two biomarkers selected from Table 1 in a plurality of cells; and ii) determining the frequency of disease or disorder of the female reproductive tract-signature cells in the plurality of cells based on the expression of the at least two biomarkers selected from Table 1.

The term “disease or disorder of the female reproductive tract”, as used herein, refers to any disease or disorder of the ovaries, the fallopian tubes, the uterus, the cervix, and/or the vagina and/or any disease or disorder that originates therefrom. In some embodiments, the disease or disorder of the female reproductive tract described herein is a non-transmissible disease or disorder of the female reproductive tract. In some embodiments, the disease or disorder of the female reproductive tract described herein is a non-transmissible disease or disorder of the female reproductive tract, wherein the symptoms include pelvic pain and/or subfertility. In some embodiments, the disease or disorder of the female reproductive tract described herein is endometriosis, ovarian cancer and/or adenomyosis. In some embodiments, the disease or disorder of the female reproductive tract described herein is endometriosis, ovarian cancer, adenomyosis and/or endometrial cancer.

The term “biomarker”, as used herein, refers to a molecule that is part of and/or generated by a cell and serves as an indicator for a disease. Often a biomarker is a gene variant or a gene product, for example an RNA or a polypeptide.

The phrase “determining a level of a biomarker”, as used herein, refers to using a nucleic acid detection technique, a peptide or protein detection technique and/or retrieval of information indicative of a level of a biomarker from a data source.

The term “subject”, as used herein, refers to a mammal, such as a mouse, guinea pig, rat, dog or human. It is understood that the preferred subject is a human.

The term “female subject”, as used herein, refers to a subject having an uterus. In some embodiments the female subject described herein is a pre-menopausal female subject. In some embodiments the female subject described herein is above 18 years old.

The term “disease or disorder of the female reproductive tract-signature cell”, as used herein, refers to a cell that is indicative of a disease or disorder of the female reproductive tract in a subject. That is, if a certain relative frequency of disease or disorder of the female reproductive tract-signature cells is determined in a plurality of cells that has been obtained from a subject, the subject is diagnosed with a disease or disorder of the female reproductive tract. Further, determining the relative frequency of disease or disorder of the female reproductive tract-signature cells in samples from the same subject at two or more time points allows to monitor the progression of the disease or disorder of the female reproductive tract in said subject.

The present invention relates to a method for the detection of a disease or disorder of the female reproductive tract with high specificity and sensitivity. Other approaches for the detection of a disease or disorder of the female reproductive tract e.g. laparoscopy or by other biomarkers, are invasive, suffer a reduced sensitivity, are not scalable and/or not suitable for early detection. The method of the present invention, on the other hand, allows measuring the level of biomarkers at the single-cell level. That is, for each cell in a plurality of cells, the level of two or more, three or more, or four or more, biomarkers is determined and based on the level of these biomarkers, it is decided for each cell if it is indicative of a disease or disorder of the female reproductive tract. The frequency of these indicative cells in a plurality of cells may then be used for determining if a subject from which the plurality of cells has been obtained, the donor, suffers from a certain medical condition and/or for determining the progression of a certain medical condition in a subject from which the plurality of cells has been obtained.

Accordingly, the invention is at least in part based on the finding that the combination of biomarkers is particularly useful for the detection of cells that are indicative of a disease or disorder of the female reproductive tract.

In certain embodiments, the invention relates to a method for determining the frequency of disease or disorder of the female reproductive tract-signature cells in a plurality of cells, the method comprising the steps of: i) determining the levels of expression of at least two biomarkers selected from Table 1 in a plurality of cells; and ii) determining the frequency of disease or disorder of the female reproductive tract-signature cells in the plurality of cells based on the expression of the at least two biomarkers selected from Table 1, wherein an increase of one or more biomarkers selected from Table 2 is indicative of disease or disorder of the female reproductive tract-signature cells and/or wherein an increase of one or more biomarkers selected from Table 3 is indicative of non-disease or disorder of the female reproductive tract-signature cells.

In certain embodiments, the invention relates to the method of the invention, wherein a)i) an alteration compared to a reference value of one or more biomarkers selected from Table 2 is indicative of an endometriosis disease state; and/or ii) an alteration of biomarker(s) selected from Table 3 is indicative of an non-endometriosis disease state; and b) wherein the reference value is indicative of a healthy status.

In certain embodiments, the invention relates to a method for determining the frequency of disease or disorder of the female reproductive tract-signature cells in a plurality of cells, the method comprising the steps of: i) determining the levels of expression of at least two biomarkers selected from Table 1 in a plurality of cells; and ii) determining the frequency of disease or disorder of the female reproductive tract-signature cells in the plurality of cells based on the expression of the at least two biomarkers selected from Table 1, wherein at least one biomarker is selected from Table 6.

The inventors found that the biomarkers from Table 6 are differentially expressed in all cycle phases. Therefore, a selection of markers from this table enables the cycle phase-independent detection of cells that are indicative of a disease or disorder of the female reproductive tract.

In certain embodiments, the invention relates to a method for determining a disease or disorder of the female reproductive tract agent signature in a sample of a female subject, the method comprising the steps of: i) determining the levels of expression of at least two biomarkers selected from Table 1 in a sample of a female subject; and ii) determining the disease or disorder of the female reproductive tract agent signature in the sample based on the expression of the at least two biomarkers selected from Table 1.

In certain embodiments, the invention relates to a method for determining an endometriosis state based on a plurality of endometrial cells, the method comprising the steps of: i) determining a frequency of cells expressing RNA of at least two biomarkers in a plurality of cells of an endometrial sample of a female subject, wherein the biomarkers are selected from Table 1; and ii) determining an endometriosis state based on the frequency determined in i).

In certain embodiments, the invention relates to the method of the invention, wherein a) i) an altered frequency of cells expressing one or more biomarkers selected from Table 2 compared to a reference frequency is indicative of an endometriosis disease state; and/or ii) an altered frequency of cells expressing of one or more biomarkers selected from Table 3 compared to a reference frequency is indicative of an non-endometriosis disease state; and b) wherein the reference value is indicative of a healthy status.

The term “disease or disorder of the female reproductive tract agent signature”, as used herein, refers to the level(s) and/or ratio(s) of biomarker(s) that is/are indicative of the disease or disorder of the female reproductive tract. As such the disease or disorder of the female reproductive tract agent signature may comprise bulk RNA, protein levels and/or data indicative of RNA or protein levels. Therefore, the sample may be processed and does not require living cells. This enables fast, standardized and robust analysis of samples.

The term “sample”, as used herein, refers to any sample, where the skilled person is aware that it may comprise a biomarker. In some embodiments, the sample described herein is a tissue sample, a lavage sample or a body fluid sample. In some embodiments, the sample described herein is a FAGS sorted tissue sample. In some embodiments, the sample described herein is an endometrial tissue sample.

The inventors found that the biomarkers represented by the signature in a sample enable the diagnosis of diseases or disorders of the female reproductive tract with high specificity and sensitivity.

In certain embodiments, the invention relates to a method for determining a disease or disorder of the female reproductive tract agent signature in a sample of a female subject, the method comprising the steps of: i) determining the levels of expression of at least two biomarkers selected from Table 1 in a sample of a female subject; and ii) determining the disease or disorder of the female reproductive tract agent signature in the sample based on the expression of the at least two biomarkers selected from Table 1, wherein an increase of one or more biomarkers selected from Table 2 is indicative of a disease or disorder of the female reproductive tract agent signature and/or wherein an increase of one or more biomarkers selected from Table 3 is non-indicative of a disease or disorder of the female reproductive tract agent signature.

In certain embodiments, the invention relates to a method for determining a disease or disorder of the female reproductive tract agent signature in a sample of a female subject, the method comprising the steps of: i) determining the levels of expression of at least two biomarkers selected from Table 1 in a sample of a female subject; and ii) determining the disease or disorder of the female reproductive tract agent signature in the sample based on the expression of the at least two biomarkers selected from Table 1, wherein at least one biomarker is selected from Table 6.

In certain embodiments, the invention relates to the method of the invention, wherein the method comprises at least one step of pre-selecting cells i) selected from the group consisting of: a) immune cells selected from the group consisting of B-cells, T-cells, dendritic cells and macrophages; b) epithelial cells selected from the group consisting of basal cells, ciliated cells and unciliated cells; c) endothelial cells; and d) smooth muscle cells; and/or ii) with at least one cell lineage marker.

In certain embodiments, the invention relates to the method of the invention, wherein the method comprises at least one step of pre-selecting cells using at least one cell lineage marker selected from the group of: a) CD14, CD16, CD45, CD15, CD11b; b) EpCAM and KRT18; and c) COL18A1, COL4A2, COL4A1, VIM or CALD1.

In certain embodiments, the invention relates to the method of the invention, wherein the method comprises at least one step of pre-selecting cells using at least one cell lineage markers CD14, CD16, CD45, CD15 and/or CD11b.

In certain embodiments, the invention relates to the method of the invention, wherein the method comprises at least one step of pre-selecting cells using at least one cell lineage markers EpCAM and/or KRT18.

In certain embodiments, the invention relates to the method of the invention, wherein the method comprises at least one step of pre-selecting cells using at least one cell lineage markers COL18A1, COL4A2, COL4A1, VIM and/or CALD1.

In certain embodiments, the invention relates to the method of the invention, wherein the sample is a proliferative phase sample.

In certain embodiments, the invention relates to the method of the invention, wherein the cells are cells obtained during the proliferative phase.

The term “proliferative phase sample”, as used herein, refers to a sample that is obtained in the proliferative phase of a female subject's menstrual cycle. In some embodiments, the proliferative phase is the first half of a menstrual cycle. In some embodiments, the proliferative phase is the phase before ovulation. Ovulation may be determined by any method known in the art, for example, based on days from the start of the menstrual cycle, based on change in vaginal secretion, based on change in progesterone levels, and/or based on body temperature.

The inventors found that during certain menstrual cycle phases the means and methods described herein are particularly sensitive and/or specific for diseases or disorders of the female reproductive tract.

In certain embodiments, the invention relates to the method of the invention, wherein the sample is a proliferative phase sample and wherein an increase of one or more biomarkers selected from Table 4 is indicative of disease or disorder of the female reproductive tract-signature cells and/or wherein an increase of one or more biomarkers selected from Table 5 is indicative of non-disease or disorder of the female reproductive tract-signature cells.

In certain embodiments, the invention relates to the method of the invention, wherein an increase of one or more biomarkers selected from Table 4 is indicative of an endometriosis disease state and/or wherein an increase of one or more biomarkers selected from Table 5 is indicative of a non-endometriosis disease state.

In certain embodiments, the invention relates to the method of the invention, wherein the sample is a proliferative phase sample and wherein at least one biomarker is selected from Table 7.

In certain embodiments, the invention relates to a method for determining the validity of an endometriosis state, the method comprising the steps of: i) determining an endometriosis state according to the invention; ii) determining or retrieving the menstrual cycle status of the subject at the time point of obtainment of the endometrial cells or of the endometrial sample; iii) determining the validity of the endometriosis state, based on the menstrual cycle status, preferably wherein the validity is considered higher if the menstrual cycle status is the proliferative phase than if the menstrual cycle status is in a different menstrual cycle state.

In certain embodiments, the invention relates to the method of the invention, wherein determining the menstrual cycle status comprises determining the RNA level of at least one menstrual cycle biomarker.

In certain embodiments, the invention relates to the method of the invention, wherein the menstrual cycle marker comprises a marker from Table 8.

The inventors found that certain markers are particularly informative during certain menstrual cycle phases and therefore enable the means and method described herein to be sensitive and/or specific for diseases or disorders of the female reproductive tract.

In certain embodiments, the invention relates to the method of the invention, wherein the method comprises at least one step of pre-selecting cells with at least one cell lineage marker, preferably using at least one cell lineage marker selected from the group of: a) ITGAM (encoding CD11b), ITGB2 (encoding CD18), CD44, FCGR3A (CD16), FCGR2A (CD32), S100A8 or S100A9; b) DRC3, RSPH3, ARMC2, LRRC23, C16orf46, ZNF487 or BBOF1; and c) COL18A1, COL4A2, COL4A1, VIM or CALD1.

The inventors found that certain cell types or cells in a certain state comprise information relevant for the diagnosis of diseases or disorders of the female reproductive tract. Therefore, selection of these cell types and/or cell states can improve the sensitivity and/or specificity of the method(s) of the invention.

Within the present invention, the level of any number of biomarkers may be determined. It is assumed that the sensitivity and specificity increase with the number of biomarkers that are used in the method of the invention. At the same time, the number of biomarkers that can be used in the method of the invention may be limited by the experimental method to determine the levels of the biomarkers and the availability of suitable binding agents.

In certain embodiments, the invention relates to the method of the invention, wherein at least 3, 4, 5, 6, 7, 8 or 9 biomarkers are determined.

The inventors found that the determination of JUP in combination with further biomarkers can increase sensitivity and/or specificity of the method described herein.

The set of biomarkers described herein may be adapted to obtain adapted panels for use in the method of the invention and to maintain high sensitivity and specificity, wherein the adapted panel consists of the same or a lower number of biomarkers by a method comprising the steps of:
i. adding one or more biomarkers to a set of biomarkers described herein to obtain an alternative panel;
ii. placing weight (e.g. as learned by CellCnn) to the biomarkers of the alternative panel by testing the alternative panel on a set of samples with known classification for a disease or disorder of the female reproductive tract associated parameter.
iii. excluding one or more biomarkers of an absolute weight below the average weight of the biomarkers of the alternative panel to obtain a provisional adapted panel;
iv. verifying the specificity and selectivity using a validation data set to identify adapted panels.

In step (i) of the method to obtain an adapted panel, the biomarker(s) to be added to the panel can be any biomarker but is/are preferably (a) biomarker(s) selected from the group listed in Table 1. In some embodiments of the invention, (one of) the biomarker(s) to be added to the panel of the current invention is known to be characteristic for a similar biologic function and/or a same cell type as one of the biomarkers of the panel of the current invention. In some embodiments of the invention, the biomarker(s) to be added to the panel may be chosen based on various reasons, including but not limited to economic reasons, availability of reagents and compatibility with the measurement equipment.

In step (ii) of the method to obtain an adapted panel, placing a weight may be done using CellCnn as described in the examples, or using any suitable weighting method known to the skilled person. The full alternative panel and/or a certain number of the biomarkers of the alternative panel can be tested to obtain information for placing a weight to the biomarkers. For example, alternative panel-minus-one controls may be used to obtain information regarding weighting (e.g., as described by Tung, James W et al. Clinics in laboratory medicine vol. 27, 3 (2007): 453-68).

In some embodiments of the invention, in step (iii) of the method to obtain an adapted panel, the biomarker with the lowest weight is excluded.

In step (iv) of the method to obtain an adapted panel, the specificity and selectivity of the provisional adapted panel may be verified as described in the examples.

Provisional adapted panels that have a specificity and selectivity below a certain specificity and selectivity threshold, are excluded.

In certain embodiments, the invention relates to the method of the invention, wherein determining the levels of expression comprises a nucleic acid detection technique.

Nucleic acid detection techniques are well known in the art (see e.g. Kolpashchikov, D. M., & Gerasimova, Y. V. (Eds.), 2013. Nucleic Acid Detection: Methods and Protocols. Humana Press.) In some embodiments, the nucleic acid detection technique described herein is at least on method selected from the group of: qPCR, ddPCR, isothermal amplification techniques, assays with visual or electric signals for point-of-care diagnostics, fluorescent in situ hybridization and signal amplification techniques.

Therefore, the biomarkers described herein may be detected on the DNA or RNA level, preferably mRNA level.

In certain embodiments the invention relates to the method of the invention, wherein the level(s) of the biomarker(s) comprise(s) a protein level.

The protein level can be determined by any method known in the art. In some embodiments, the protein level described herein is determined by an antibody-based assay. That is, any assay that comprises the use of antibodies and is suitable for determining the expression level of a biomarker may be used in the present invention. Preferably, antibodies are used that bind directly to the biomarker. Within the present invention, the antibodies are preferably labeled to facilitate detection and/or quantification of a biomarker. For example, antibodies may be labeled with a fluorophore to allow detection and/or quantification of biomarkers in flow cytometry-based assays or metal isotopes to allow detection and/or quantification of biomarkers in mass cytometry-based assays. In some embodiments, the invention relates to the method according to the invention, wherein the antibody-based assay is an antibody-based flow cytometry or mass cytometry assay. In some embodiments, the protein level described herein is determined by ELISA, preferably multiplexed ELISA.

In certain embodiments, the invention relates to the method of the invention, wherein the plurality of cells are primary cells or wherein the sample is a primary sample.

The ter “primary sample”, as used herein, refers to any sample that is not cultured for cell expansion. The primary sample described herein may nevertheless be stored, maintained or processed.

The inventors found that the methods described herein do not require cell culture mediated cell expansion to be specific and/or sensitive. This enables the method to be more efficient than previous methods.

In certain embodiments, the invention relates to the method of the invention, wherein the levels are determined in an endometrium sample, menstrual blood sample, vaginal smear sample and/or a cervical smear sample.

In certain embodiments, the invention relates to the method of the invention, wherein the levels are determined in a blood sample, such as a plasma or serum sample.

The inventors found that method of the invention is particularly sensitive, specific and or minimally invasive when using certain types of samples.

In certain embodiments, the invention relates to the method of the invention, wherein the method additionally comprises determining at least one non-molecular marker, preferably wherein the non-molecular marker comprises a marker selected from the group consisting of: age, weight, BMI, gravidity, parity, ethnicity, fertility status, previous laparoscopies, previous use of medication and other gynaecological disorders.

In certain embodiments, the invention relates to the method of the invention, wherein the method additionally comprises determining or retrieving at least one non-molecular marker, preferably wherein the non-molecular marker comprises a marker selected from the group consisting of: age, weight, BMI, gravidity, parity, ethnicity, fertility status, previous laparoscopies, previous use of medication and other gynaecological disorders.

The term “other gynaecological disorder”, as used herein, refers to any gynaecological disorder other than the disease or disorder of the female reproductive tract that is diagnosed, predicted and/or classified according to the method of the invention. In some embodiments, the other gynaecological disorder described herein is a gynaecological disorder other than endometriosis and ovarian cancer. In some embodiments, the other gynaecological disorder described herein is a gynaecological disorder other than endometriosis. In some embodiments, the other gynaecological disorder described herein is a gynaecological disorder other than ovarian cancer.

The inventors found that non-molecular markers can improve the sensitivity and/or specificity of the method of the invention.

In certain embodiments, the invention relates to the method of the invention, wherein the method is at least partially computer-implemented and wherein the levels of expression are determined by retrieving data indicative for the levels of expression.

The inventors found that the method of the invention can be used on databases and/or data of samples. This enables scalability and/or separating the sample obtainment procedure from the interpretation of the sample.

In certain embodiments, the invention relates to a method for prediction of disease development, disease progression and/or disease outcome of a female subject having a disease or disorder of the female reproductive tract or at risk of having a disease or disorder of the female reproductive tract, the method comprising the steps of: a) i) determining the frequency of disease or disorder of the female reproductive tract-signature cells in a sample of a female subject according to the method of the invention; and/or ii) determining a disease or disorder of the female reproductive tract agent signature in a sample of a female subject according to the method of the invention; b) comparing the frequency determined in a)i) and/or the agent signature determined in a)ii) to a prediction reference pattern; and c) predicting disease development, disease progression and/or disease outcome of the female subject based on the comparison in step b).

In certain embodiments, the invention relates to a method for prediction of disease outcome, disease development and/or disease progression of a female subject having endometriosis or at risk of having a endometriosis, the method comprising the steps of: a) determining an endometriosis state according to the method of the invention; b) comparing the endometriosis state determined in a) to a prediction reference pattern; and c) predicting disease outcome disease development and/or disease progression of the female subject based on the comparison in step b).

The phrase “risk of having a disease or disorder of the female reproductive tract”, as used herein, refers to having a risk factor for a disease or disorder of the female reproductive tract and/or at least one symptom of a disease or disorder of the female reproductive tract.

The term “reference pattern”, as used herein, refers to a predetermined pattern or a predetermined datapoint that can be used for comparison and is preferably obtained from reference subjects. The reference pattern comprises at least one datapoint, such as a datapoint that can be used as a threshold. In some embodiments, the reference pattern is a machine learning model.

In certain embodiments, the invention relates to the method of the invention, wherein 1.) an increased frequency of disease or disorder of the female reproductive tract-signature cells expressing biomarkers from Table 2 compared to the reference pattern; and/or 2.) an increased level of the biomarkers from Table 2 in the disease or disorder of the female reproductive tract agent signature compared to the reference pattern is indicative for more likely disease development, more likely disease progression and/or worsening of disease outcome.

In certain embodiments, the invention relates to the method of the invention, wherein 1.) determining an increased frequency of cells expressing the biomarker(s) from Table 2 compared to the prediction reference pattern; and/or 2.) determining an increased level of the biomarker(s) from Table 2 compared to the reference pattern is indicative for worsening of disease outcome more likely disease development and/or more likely disease progression.

In certain embodiments, the invention relates to the method of the invention, wherein 1.) an increased frequency of disease or disorder of the female reproductive tract-signature cells expressing biomarkers from Table 3 compared to the reference pattern; and/or 2.) an increased level of the biomarkers from Table 3 in the disease or disorder of the female reproductive tract agent signature compared to the reference pattern is indicative for less likely disease development, less likely disease progression and/or improvement of disease outcome.

In certain embodiments, the invention relates to the method of the invention, wherein 1.) determining an increased frequency of cells expressing the biomarker(s) from Table 3 compared to the prediction reference pattern; and/or 2.) determining an increased level of one or more biomarkers from Table 3 compared to the reference pattern is indicative for improvement of disease outcome less likely disease development and/or less likely disease progression.

In certain embodiments the invention relates to a method for diagnosing a female subject with a disease or disorder of the female reproductive tract, the method comprising the steps of: a) i) determining the frequency of disease or disorder of the female reproductive tract-signature cells in a sample of a female subject according to the method of the invention; and/or ii) determining a disease or disorder of the female reproductive tract agent signature in a sample of a female subject according to the method of the invention; b) comparing the frequency determined in a)i) and/or the agent signature determined in a)ii) to a diagnosis reference pattern; and c) diagnosing a female subject with a disease or disorder of the female reproductive tract based on the comparison in step b).

In certain embodiments, the invention relates to a method for predicting susceptibility to a treatment for endometriosis of a female subject having endometriosis or at risk of having endometriosis, the method comprising the steps of: a) determining an endometriosis state according to the method of the invention; b) comparing the endometriosis state to a susceptibility reference pattern; and c) predicting susceptibility to a treatment for endometriosis of the female subject based on the comparison in step b).

In certain embodiments the invention relates to a method for monitoring a female subject for a disease or disorder of the female reproductive tract, the method comprising the steps of: a) at a first time point: i) determining the frequency of disease or disorder of the female reproductive tract-signature cells in a sample of a female subject according to the method of the invention; and/or ii) determining a disease or disorder of the female reproductive tract agent signature in a sample of a female subject according to the method of the invention; b) at a second time point: i) determining the frequency of disease or disorder of the female reproductive tract-signature cells in a sample of a female subject according to the method of the invention; and/or ii) determining a disease or disorder of the female reproductive tract agent signature in a sample of a female subject according to the method of the invention; c) comparing the frequency determined in a)i) and/or b)i) and/or the agent signature determined in a)ii) and/or b)ii) to a monitoring reference pattern, wherein the first time point is combined with the second time point.; and d) monitoring a female subject based on the comparison in step b).

In some embodiments, the invention relates to the method for monitoring described herein or the method(s) for diagnosis described herein, wherein the method is used as a screening method in healthy female subjects to detect disease development.

In certain embodiments, the invention relates to a method for predicting susceptibility to a treatment for a disease or disorder of the female reproductive tract of a female subject having a disease or disorder of the female reproductive tract or at risk of having a disease or disorder of the female reproductive tract, the method comprising the steps of: a) i) determining a frequency of disease or disorder of the female reproductive tract-signature cells in a sample of a female subject according to the method of the invention; and/or ii) determining a disease or disorder of the female reproductive tract agent signature in a sample of a female subject according to the method of the invention; b) comparing the frequency determined in a)i) and/or the signature determined in a)ii) to a susceptibility reference pattern; and c) predicting susceptibility to a treatment for a disease or disorder of the female reproductive tract of the female subject based on the comparison in step b).

In certain embodiments, the invention relates to a method for predicting susceptibility to a treatment for a disease or disorder of the female reproductive tract of a female subject having a disease or disorder of the female reproductive tract or at risk of having a disease or disorder of the female reproductive tract, the method comprising the steps of: a) i) determining a frequency of disease or disorder of the female reproductive tract-signature cells in a sample of a female subject according to the method of the invention; and/or ii) determining a disease or disorder of the female reproductive tract agent signature in a sample of a female subject according to the method of the invention; b) comparing the frequency determined in a)i) and/or the signature determined in a)ii) to a susceptibility reference pattern; and c) predicting susceptibility to a treatment for a disease or disorder of the female reproductive tract of the female subject based on the comparison in step b), wherein a frequency of disease or disorder of the female reproductive tract-signature cells and/or a disease or disorder of the female reproductive tract agent signature above the susceptibility reference pattern is indicative of a subject being susceptible to a treatment.

In some embodiments, the susceptibility to a treatment described herein, is the disease outcome and/or disease progression after treatment. For example, the method of the invention can be used to predict disease recurrence after the laparoscopic removal of endometriosis lesions.

In certain embodiments the invention relates to the method of the invention, wherein the treatment for a disease or disorder of the female reproductive tract is an anti-cancer treatment. In some embodiments, the anti-cancer treatment described herein is at least one compound selected from the group of carboplatin, avastin, paclitaxel, doxil, methotrexate, lynparza, adriamycin, gemzar, olaparib, doxorubicin, alkeran, paraplatin, zejula, bevacizumab, cisplatin, doxorubicin, gemcitabine, rubraca, cosmegen, hycamtin, topotecan, cyclophosphamide, melphalan, toposar and etopophos.

In certain embodiments the invention relates to the method of the invention, wherein the treatment for a disease or disorder of the female reproductive tract is an endometriosis treatment selected from the group of: hormonal treatment, physiotherapy, surgery, multimodal pain therapy (drugs (e.g. Targin, Oxynorm), TENS machine), individual nutritional counselling (e.g. eating less meat) and complementary medicine.

In certain embodiments, the invention relates to the method of the invention wherein the treatment for a disease or disorder of the female reproductive tract is a treatment selected from the group of: pain medication, hormonal therapy, fertility treatment and surgery.

In certain embodiments, the invention relates to the method of the invention wherein the treatment for endometriosis is a treatment selected from the group consisting of: pain medication, hormonal therapy, fertility treatment and surgery.

The term “pain medication”, as used herein, refers to any pain medication that is used to treat symptoms of endometriosis (see e.g. Ruhland, B., et al., 2011, Minerva Ginecol 63: 1-2). In some embodiments, the pain medication described herein is a pain medication selected from the group of ibuprofen, naproxen and oxycodone.

The term “hormonal therapy”, as used herein, refers to any hormonal therapy that is used to treat symptoms of endometriosis (see e.g. Ruhland, B., et al., 2011, Minerva Ginecol 63: 1-2). In some embodiments, the hormonal therapy described herein is progesterone treatment, preferably a gestagen selected from the group of Desogestrel, Dienogest, Levonorgestrel. The hormonal therapy described herein may be administered orally, by implantation, by injection, transdermal or using an intrauterine device.

The term “fertility treatment”, as used herein, refers to any fertility treatment that is used to treat infertility or subfertility in the context of endometriosis (see e.g. Becker, C. M., Gattrell, W. T., Gude, K., & Singh, S. S., 2017, Fertility and sterility, 108(1), 125-136). In some embodiments, the fertility treatment described herein is in vitro fertilization. In some embodiments, the fertility treatment described herein is a treatment selected from the group of: clomiphene citrate, gonadotropins, metformin, letrozole, bromocriptine, follitropin alpha, Intrauterine insemination (IUI) and in vitro fertilization (IVF).

The term “surgery”, as used herein, refers to any surgical procedure that is used to treat symptoms of endometriosis (see e.g. Leonardi, M., et al. 2020, Journal of minimally invasive gynecology, 27(2), 390-407.). In some embodiments, the surgery described herein is a form of surgery selected from the group of conservative surgery, complex surgery, radical surgery and Laparoscopy.

The choice of treatment is particularly relevant in endometriosis, because it can have an effect on disease progression and/or fertility that can be irreversible.

Accordingly, the invention is at least in part based on the finding that the methods described herein enable accurate prediction of susceptibility to a treatment.

In certain embodiments, the invention relates to the method of the invention, wherein the susceptibility reference pattern or the prediction reference pattern is obtained from reference subjects, wherein at least one of the reference subjects has been diagnosed with the disease or disorder of the female reproductive tract.

In certain embodiments, the invention relates to the method of the invention, wherein the susceptibility reference pattern or the prediction reference pattern is obtained from reference subjects, wherein at least one of the reference subjects has been diagnosed with endometriosis.

The inventors found that using data of subjects suffering from a disease or disorder of the female reproductive tract can be used as a reference.

Accordingly, the invention is at least in part based on the finding that data from diseased subjects is particularly useful for the reference pattern in the methods described herein.

In certain embodiments, the invention relates to the method of the invention, wherein obtaining the susceptibility reference pattern or the prediction reference pattern from reference subjects comprises a machine-learning technique.

The term “machine-learning technique”, as used herein, refers to a computer-implemented technique that enables automatic learning and/or improvement from an experience (e.g., training data and/or obtained data) without the necessity of explicit programming of the lesson learned and/or improved. In some embodiments, the machine learning technique comprises at least one technique selected from the group of Logistic regression, CART, Bagging, Random Forest, Gradient Boosting, Linear Discriminant Analysis, Gaussian Process Classifier, Gaussian NB, Linear, Lasso, Ridge, ElasticNet, partial least squares, KNN, DecisionTree, SVR, AdaBoost, GradientBoost, neural net and ExtraTrees.

The inventors found that machine-learning techniques provide an efficient and/or unbiased way to identify patterns predictive for disease- and treatment-related parameters.

In certain embodiments, the invention relates to the method of the invention, wherein obtaining the susceptibility reference pattern or the prediction reference pattern from reference subjects comprises a convolutional neural network and/or logistic regression.

In certain embodiments, the invention relates to the method of the invention, wherein obtaining the susceptibility reference pattern or the prediction reference pattern from reference subjects comprises a machine-learning technique, preferably a convolutional neural network and/or logistic regression.

The CellCnn convolutional neural network has been described previously (Arvaniti, E., Claassen, M., 2017, Nat Commun 8, 14825; Bodenmiller et al., Nat Biotechnol, 2012, 30(9), 858-867; Amir et al., Nat Biotechnol, 2013, 31(5), 545-552; Levine et al., Cell, 2015, 162(1), 184-197; Horowitz et al., Sci Transl Med, 2013, 5(208), 208ra145) and is publicly available (https://github.com/eiriniar/CellCnn). Further, it is described in the Examples how the CellCnn convolutional neural network may be used in the context of the invention.

In certain embodiments, the invention relates to a method for classification of a female subject having a disease or disorder of the female reproductive tract or at risk of having a disease or disorder of the female reproductive tract into a class, the method comprising the steps of: a. i) determining a frequency of disease or disorder of the female reproductive tract-signature cells in a sample of a female subject according to the method of the invention; ii) determining a disease or disorder of the female reproductive tract agent signature in a sample of a female subject according to the method of the invention; iii) predicting a disease development, disease progression and/or disease outcome of a female subject according to the method of the invention; and/or iv) predicting susceptibility to a treatment for a disease or disorder of the female reproductive tract of a female subject according to the method of the invention; and b. classifying the female subject according to the frequency determined in i), agent signature determined in ii), prediction of iii), and/or prediction in iv).

In certain embodiments, the invention relates to a method for classification of a female subject having a endometriosis or at risk of having a endometriosis into a class, the method comprising the steps of: a)i) determining an endometriosis state according to the method of the invention; ii) predicting a disease outcome, disease development and/or disease progression of a female subject according to the method of the invention; and/or iii) predicting susceptibility to a treatment for endometriosis of a female subject according to the method of the invention; and b) classifying the female subject according to the frequency determined in i), signature determined in ii), prediction of iii), and/or prediction in iv).

In certain embodiments, the invention relates to the method of the invention, wherein at least one class is indicative of the stage and/or severity of the endometriosis.

The term “stage of the endometriosis”, as used herein, refers to an established stage of endometriosis such as the four rASRM stages (Rock, J. A., & ZOLADEX Endometriosis Study Group, 1995, Fertility and sterility, 63(5), 1108-1110) or the or ENZIAN stages P1-3, O1-3, T1-3, A1-3, B1-3, C1-3, F(Location).

In certain embodiments, the invention relates to a composition comprising reagents for the detection of biomarkers for the diagnosis of a disease or disorder of the female reproductive tract, the biomarkers comprising or consisting of at least two markers from Table 1.

In certain embodiments, the invention relates to a composition comprising reagents for the detection of biomarkers for the diagnosis of endometriosis, the biomarkers comprising or consisting of at least two markers from Table 1.

In certain embodiments, the invention relates to a pharmaceutical product comprising a compound against a disease or disorder of the female reproductive tract for use in treatment of a female subject that is predicted as susceptible to a treatment for a disease or disorder of the female reproductive tract according to the method of the invention.

The term “pharmaceutical product”, as used herein, refers to a preparation which is in such form as to permit the biological activity of an active ingredient contained therein to be effective, and which contains no additional components which are unacceptably toxic to a subject to which the formulation would be administered.

The term “compound against a disease or disorder of the female reproductive tract”, as used herein, refers to any compound that is known to be effective in the treatment of disease or disorder of a female reproductive tract and/or symptoms thereof.

The inventors found that, using the method(s) of the invention, subject populations that are particularly sensitive to certain pharmaceutical products can be identified. As such, the pharmaceutical products have a surprisingly enhanced risk/benefit ratio in this/these subject population(s).

In certain embodiments the invention relates to the pharmaceutical product of the invention, wherein the compound against a disease or disorder of the female reproductive tract is an anti-cancer treatment.

In certain embodiments, the invention relates to the pharmaceutical product of the invention, wherein the compound against a disease or disorder of the female reproductive tract is selected from the group of: ibuprofen, naproxen, oxycodone, desogestrel, dienogest, levonorgestrel, clomiphene citrate, gonadotropins, metformin, letrozole and bromocriptine.

In certain embodiments the invention relates to a method of treatment, the method comprising the steps of: 1) classifying and/or predicting susceptibility to a treatment for a disease or disorder of the female reproductive tract of a female subject having a disease or disorder of the female reproductive tract or at risk of having a disease or disorder of the female reproductive tract according to the method of the invention; and 2) treating the female subject with a treatment for a disease or disorder of the female reproductive tract, wherein the choice of a treatment for a disease or disorder of the female reproductive tract depends on the predicted susceptibility and/or the classification of susceptibility in step (1).

In certain embodiments the invention relates to a method of treatment, the method comprising the steps of: 1) classifying and/or predicting susceptibility to a treatment for a disease or disorder of the female reproductive tract of a female subject having a disease or disorder of the female reproductive tract or at risk of having a disease or disorder of the female reproductive tract according to the method of the invention; and 2) treating the female subject with at least one disease or disorder of the female reproductive tract treatment selected from the group of anti-cancer treatment, pain medication, hormonal therapy, fertility treatment and surgery, wherein the choice of a disease or disorder of the female reproductive tract treatment depends on the predicted susceptibility and/or the classification of susceptibility in step (1).

In certain embodiments the invention relates to a method of treatment, the method comprising the steps of: 1) classifying and/or predicting susceptibility to a treatment for a disease or disorder of the female reproductive tract of a female subject having a disease or disorder of the female reproductive tract or at risk of having a disease or disorder of the female reproductive tract according to the method of the invention; and 2) treating the female subject with a pharmaceutical product selected from the group of: ibuprofen, naproxen, oxycodone, desogestrel, dienogest, levonorgestrel, clomiphene citrate, gonadotropins, metformin, letrozole and bromocriptine, wherein the choice of pharmaceutical product depends on the predicted susceptibility and/or the classification of susceptibility in step (1).

In certain embodiments, the invention relates to the method of the invention, the composition of the invention or the pharmaceutical product of the invention, wherein the disease or disorder of the female reproductive tract is endometriosis, ovarian cancer and/or adenomyosis.

In certain embodiments, the invention relates to the method of the invention, the composition of the invention or the pharmaceutical product of the invention, wherein the disease or disorder of the female reproductive tract is endometriosis, ovarian cancer, adenomyosis and/or endometrial cancer.

The term “ovarian cancer”, as used herein, refers to a condition characterized by anomalous rapid proliferation of ovarian cells and/or of cells in the ovarian area of a subject. In some embodiments, the ovarian cancer described herein is a primary ovarian cancer.

The term “adenomyosis”, as used herein, refers to a condition characterized by cell growth within the uterus characterized by cell growth that causes the uterus to thicken and/or enlarge.

The term “endometrial cancer”, as used herein, refers to a condition characterized by anomalous rapid proliferating cells in the tissue lining the uterus. In some embodiments, the endometrial cancer described herein is a primary endometrial cancer.

The inventors found that the means and methods of the invention are particularly sensitive and/or specific in the context of endometriosis, ovarian cancer, adenomyosis and/or in distinguishing between such indications.

In certain embodiments, the invention relates to the method of the invention, the composition of the invention or the pharmaceutical product of the invention, wherein the disease or disorder of the female reproductive tract is endometriosis.

The term “endometriosis”, as used herein, refers to a disease of the female reproductive system in which cells similar to those in the endometrium, the layer of tissue that normally covers the inside of the uterus, grow outside the uterus.

Risk factors for endometriosis include without limitation genetic risk factors (e.g. a relative diagnosed with endometriosis and/or a mutation in one or more of the WNT4, GREB1/FN1, ID4, 7p15.2, CDKN2BAS, 10q26, VEZT, MUC16 genes/regions), a history of symptoms of endometriosis and environmental toxins (e.g. exposure to estrogen, exposure to dioxin or obstruction of menstrual outflow).

The inventors found that the means and methods of the invention are particularly sensitive and/or specific in the context of endometriosis.

In certain embodiments, the invention relates to the method of the invention, the composition of the invention or the pharmaceutical product of the invention, wherein the endometriosis is selected from the group of: peritoneal endometriosis, endometriomas, deeply infiltrating endometriosis, tubal endometriosis and abdominal wall endometriosis.

The inventors found that the biomarkers described herein are particularly altered in certain types of endometriosis. Accordingly, the invention is at least in part based on the finding that the methods described herein are particularly sensitive and/or specific in certain types of endometriosis.

In certain embodiments the invention relates to the method of the invention, the composition of the invention or the pharmaceutical product of the invention, wherein the endometriosis is at rASRM I stage. In certain embodiments the invention relates to the method of the invention, the composition of the invention or the pharmaceutical product of the invention, wherein the endometriosis is at rASRM II stage. In certain embodiments the invention relates to the method of the invention, the composition of the invention or the pharmaceutical product of the invention, wherein the endometriosis is at rASRM III stage. In certain embodiments the invention relates to the method of the invention, the composition of the invention or the pharmaceutical product of the invention, wherein the endometriosis is at rASRM IV stage. In certain embodiments the invention relates to the method of the invention, the composition of the invention or the pharmaceutical product of the invention, wherein the endometriosis is at rASRM III or IV stage. In certain embodiments the invention relates to the method of the invention, the composition of the invention or the pharmaceutical product of the invention, wherein the endometriosis is at rASRM II or III stage. In certain embodiments the invention relates to the method of the invention, the composition of the invention or the pharmaceutical product of the invention, wherein the endometriosis is at rASRM I or II stage. In certain embodiments the invention relates to the method of the invention, the composition of the invention or the pharmaceutical product of the invention, wherein the endometriosis is at rASRM I, II or III stage.

In certain embodiments, the invention relates to the method of the invention, the composition of the invention or the pharmaceutical product of the invention, wherein the endometriosis at rASRM II, III, or IV stage.

The inventors found that the biomarkers described herein are particularly altered in later stages of endometriosis.

Accordingly, the invention is at least in part based on the finding that the methods described herein are particularly sensitive and/or specific in later stages of endometriosis.

In certain embodiments, the invention relates to a computer program product comprising instructions to execute the method of the invention, wherein the method is computer-implemented.

The computer program product described herein may comprise computer-readable program instructions that can be downloaded to respective computing/processing devices from a computer-readable storage medium or to an external computer or external storage device via a network.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages.

The computer-readable program instructions may execute entirely on the user's computer, partly on the user's computer, as a stand-alone software package, partly on the user's computer and partly on a remote computer or entirely on the remote computer or server.
“a,” “an,” and “the” are used herein to refer to one or to more than one (i.e., to at least one, or to one or more) of the grammatical object of the article.
“or” should be understood to mean either one, both, or any combination thereof of the alternatives.
“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

The terms “about” or “approximately”, as used herein, refer to “within 20%”, more preferably “within 10%”, and even more preferably “within 5%”, of a given value or range.

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention further relates to the following items:
1. A method for determining the frequency of disease or disorder of the female reproductive tract-signature cells in a plurality of cells, the method comprising the steps of:
   i) determining the levels of expression of at least two biomarkers selected from Table 1 in a plurality of cells; and
   ii) determining the frequency of disease or disorder of the female reproductive tract-signature cells in the plurality of cells based on the expression of the at least two biomarkers selected from Table 1, preferably wherein an increase of one or more biomarkers selected from Table 2 is indicative of disease or disorder of the female reproductive tract-signature cells and/or wherein an increase of one or more biomarkers selected from Table 3 is indicative of non-disease or disorder of the female reproductive tract-signature cells.
2. A method for determining a disease or disorder of the female reproductive tract agent signature in a sample of a female subject, the method comprising the steps of:
   i) determining the levels of expression of at least two biomarkers selected from Table 1 in a sample of a female subject; and
   ii) determining the disease or disorder of the female reproductive tract agent signature in the sample based on the expression of the at least two biomarkers selected from Table 1, preferably wherein an increase of one or more biomarkers selected from Table 2 is indicative of a disease or disorder of the female reproductive tract agent signature and/or wherein an increase of one or more biomarkers selected from Table 3 is non-indicative of a disease or disorder of the female reproductive tract agent signature.
3. The method of items 1 or 2, wherein the sample is a proliferative phase sample, preferably wherein an increase of one or more biomarkers selected from Table 4 is indicative of disease or disorder of the female reproductive tract-signature cells and/or wherein an increase of one or more biomarkers selected from Table 5 is indicative of non-disease or disorder of the female reproductive tract-signature cells.
4. The method of any one of items 1 to 3, wherein the method comprises at least one step of pre-selecting cells with at least one cell lineage marker, preferably using at least one cell lineage marker selected from the group of:
   a) ITGAM (encoding CD11b), ITGB2 (encoding CD18), CD44, FCGR3A (CD16), FCGR2A (CD32), S100A8 or S100A9;
   b) DRC3, RSPH3, ARMC2, LRRC23, C16orf46, ZNF487 or BBOF1; and
   c) COL18A1, COL4A2, COL4A1, VIM or CALD1.
5. The method of any one of items 1 to 4, wherein at least 3, 4, 5, 6, 7, 8 or 9 biomarkers are determined.
6. The method of any one of items 1 to 5, wherein determining the levels of expression comprises a nucleic acid detection technique.
7. The method of any one of items 1 to 6, wherein the plurality of cells are primary cells or wherein the sample is a primary sample.
8. The method of any one of items 1 to 7, wherein the levels are determined in an endometrium sample, menstrual blood sample, vaginal smear sample and/or a cervical smear sample.
9. The method of any one of items 1 to 7, wherein the levels are determined in a blood sample, such as a plasma or serum sample.
10. The method of any one of items 1 to 9, wherein the method additionally comprises determining at least one non-molecular marker, preferably wherein the non-molecular marker comprises a marker selected from the group consisting of: age, weight, BMI, gravidity, parity, ethnicity, fertility status, previous laparoscopies, previous use of medication and other gynaecological disorders.
11. The method of any one of items 1 to 10, wherein the method is at least partially computer-implemented and wherein the levels of expression are determined by retrieving data indicative for the levels of expression.
12. A method for prediction of disease development, disease progression and/or disease outcome of a female subject having a disease or disorder of the female reproductive tract or at risk of having a disease or disorder of the female reproductive tract, the method comprising the steps of:
   a) i) determining the frequency of disease or disorder of the female reproductive tract-signature cells in a sample of a female subject according to the method of any one of items 1, 3 to 11; and/or
      ii) determining a disease or disorder of the female reproductive tract agent signature in a sample of a female subject according to the method of any one of items 2 to 11;
   b) comparing the frequency determined in a)i) and/or the agent signature determined in a)ii) to a prediction reference pattern; and
   c) predicting disease development, disease progression and/or disease outcome of the female subject based on the comparison in step b).
13. The method of item 12, wherein
   1.) an increased frequency of disease or disorder of the female reproductive tract-signature cells expressing biomarkers from Table 2 compared to the reference pattern; and/or
   2.) an increased level of the biomarkers from Table 2 in the disease or disorder of the female reproductive tract agent signature compared to the reference pattern is indicative for more likely disease development, more likely disease progression and/or worsening of disease outcome.
14. The method of item 12 or 13, wherein
   1.) an increased frequency of disease or disorder of the female reproductive tract-signature cells expressing biomarkers from Table 3 compared to the reference pattern; and/or
   2.) an increased level of the biomarkers from Table 3 in the disease or disorder of the female reproductive tract agent signature compared to the reference pattern
   is indicative for less likely disease development, less likely disease progression and/or improvement of disease outcome.
15. A method for predicting susceptibility to a treatment for a disease or disorder of the female reproductive tract of a female subject having a disease or disorder of the female reproductive tract or at risk of having a disease or disorder of the female reproductive tract, the method comprising the steps of:
   a) i) determining a frequency of disease or disorder of the female reproductive tract-signature cells in a sample of a female subject according to the method of any one of items 1, 3 to 11; and/or
   ii) determining a disease or disorder of the female reproductive tract agent signature in a sample of a female subject according to the method of any one of items 2 to 11;
b) comparing the frequency determined in a)i) and/or the signature determined in a)ii) to a susceptibility reference pattern; and
c) predicting susceptibility to a treatment for a disease or disorder of the female reproductive tract of the female subject based on the comparison in step b), preferably wherein a frequency of disease or disorder of the female reproductive tract-signature cells and/or a disease or disorder of the female reproductive tract agent signature above the susceptibility reference pattern is indicative of a subject being susceptible to a treatment.
16. The method of item 15 wherein the treatment for a disease or disorder of the female reproductive tract is a treatment selected from the group of: pain medication, hormonal therapy, fertility treatment and surgery.
17. The method of any one of items 12 to 16, wherein the susceptibility reference pattern or the prediction reference pattern is obtained from reference subjects, wherein at least one of the reference subjects has been diagnosed with the disease or disorder of the female reproductive tract.
18. The method of item 17, wherein obtaining the susceptibility reference pattern or the prediction reference pattern from reference subjects comprises a machine-learning technique, preferably a convolutional neural network and/or logistic regression.
19. A method for classification of a female subject having a disease or disorder of the female reproductive tract or at risk of having a disease or disorder of the female reproductive tract into a class, the method comprising the steps of:
   a. i) determining a frequency of disease or disorder of the female reproductive tract-signature cells in a sample of a female subject according to the method of any one of items 1, 3 to 11;
      ii) determining a disease or disorder of the female reproductive tract agent signature in a sample of a female subject according to the method of any one of items 2 to 11;
      iii) predicting a disease development, disease progression and/or disease outcome of a female subject according to the method of any one of items 12 to 14, 17 or 18; and/or
      iv) predicting susceptibility to a treatment for a disease or disorder of the female reproductive tract of a female subject according to the method of any one of items 15 to 18; and
   b. classifying the female subject according to the frequency determined in i), agent signature determined in ii), prediction of iii), and/or prediction in iv).
20. The method of item 19, wherein at least one class is indicative of the stage and/or severity of the endometriosis.
21. A composition comprising reagents for the detection of biomarkers for the diagnosis of a disease or disorder of the female reproductive tract, the biomarkers comprising or consisting of at least two markers from Table 1.
22. A pharmaceutical product comprising a compound against a disease or disorder of the female reproductive tract for use in treatment of a female subject that is predicted as susceptible to a treatment for a disease or disorder of the female reproductive tract according to the method of item 15 to 18.
23. The pharmaceutical product of item 22, wherein the compound against a disease or disorder of the female reproductive tract is selected from the group of: ibuprofen, naproxen, oxycodone, desogestrel, dienogest, levonorgestrel, clomiphene citrate, gonadotropins, metformin, letrozole and bromocriptine.
24. The method of items 1 to 20, the composition of item 21 or the pharmaceutical product of item 22 or 23, wherein the disease or disorder of the female reproductive tract is endometriosis, ovarian cancer and/or adenomyosis.
25. The method of item 24, the composition of item 24 or the pharmaceutical product of item 24, wherein the disease or disorder of the female reproductive tract is endometriosis.
26. The method of the item 25, the composition of item 25 or the pharmaceutical product of item 25, wherein the endometriosis is selected from the group of: peritoneal endometriosis, endometriomas, deeply infiltrating endometriosis, tubal endometriosis and abdominal wall endometriosis.
27. The method of item 25 or 26, the composition of item 25 or 26 or the pharmaceutical product of items 25 or 26, wherein the endometriosis at rASRM II, III, or IV stage.
28. A computer program product comprising instructions to execute the method of any one of items 11 to 20, 24 to 27, wherein the method is computer-implemented.

### BRIEF DESCRIPTION OF FIGURES

FIG. 1. ROC curves of three learners of the proliferative phase with AUC=1.00 (A), 1.00 (B), 0.78 (C)

FIG. 2. ROC curves of representative learners of all cycle phases with AUC=0.83 (A), 0.79 (B), 0.95 (C).

FIG. 3. Expression levels of 6 selected genes from the analysis of differentially expressed genes between endometriosis and non-endometriosis samples of all cycle phases (A-F) or the proliferative phase (G-L) FIG. 4. Top 50 GO terms and pathways for the gene signature-ranked by the adjusted p-value. Indicated are the number of genes involved in each GO term and pathway (intersection_size) and the −log 10 p-value.

FIG. 5. UMAPs highlighting the cell-type of interest which express the signature for samples in the Proliferative phase (A) and samples from Proliferative and Secretory Phase (B).

FIG. 6. Shown is the AUC for the trained models for the inventors signature (A, median AUC=0.78), competitive signature (B, median AUC=0.56) and the combined gene list from competitive and inventors, respectively (C, median AUC=0.78).

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1

### Clinical Study Design

Phase I Discovery: An open label study for the discovery of biomarkers for the diagnosis and prognosis of endometriosis.

### Study Population

Patients were recruited at the Frauenklinik, Bern after approval of this application. Inclusion criteria for this study include women who provide Informed Consent and are scheduled for laparoscopic surgery for reasons including suspected endometriosis, tubal ligation, idiopathic infertility or other gynaecological pathologies as part of their planned clinical treatment. Women above 18 years old of all ethnicities and sociodemographic backgrounds were included. Patients with other pre-existing inflammatory diseases, pregnancy, malignancy or undergoing emergency surgery were excluded.

Blood and/or endometrial biopsy were isolated from a total of 256 patients with suspected endometriosis immediately before surgery.

### Clinical Investigation Objectives

### Primary:

The primary objective of this project is to identify a significant biomarker signature in tissue (endometrial biopsies, ectopic lesions, and peritoneal fluid) of women with or without endometriosis, which contributes to identifying patients suffering from endometriosis.

### Secondary:

The secondary objectives of this projects are to identify:
i) the biological differences in the tissue between women with variations in endometriosis-associated symptoms;
ii) the biological differences in the tissue between women with variations in endometriosis-associated treatment outcomes;
iii) The above biological differences observed in the tissue will serve as basis for the development and/or the evaluation of potential drugs.

### Data Types

Clinical Parameters: Full, anonymized clinical data e.g. age, weight, BMI, gravidity and parity, ethnicity, previous laparoscopies and use of (hormonal) medication and other gynaecological disorders, etc.

Single-cell RNA sequencing: RNA expression profile performed on Endometrial biopsy (Pipelle) from 42 patients.

### Inclusion Criteria

Signed and dated informed consent.

Age: Pre-menopausal women >18 years.

Women undergoing laparoscopic surgery.

Good general health as proven by medical history, physical and gynecological examinations, and laboratory test results.

The study was performed on patients in the proliferative or secretory phase of the menstrual cycle. Patients had progesterone levels measured prior to the operation and endometrial biopsies were analysed by a pathologist to confirm the menstrual cycle phase.

The planned study has no influence on further therapeutic steps.

### Exclusion Criteria

Patients unlikely to cooperate or legally incompetent, including patients who are institutionalized by court or official order. Any condition which could interfere with the patient's ability to comply with the study.

Patients who are pregnant or lactating.

Patients using any anti-inflammatory drugs or suffering from other inflammatory diseases.

Patients with hormonal treatment or IUD less than 3 months before the operation were excluded.

Exclusion of patients with no definite diagnosis of endometriosis by histology.

Patients with a menstrual cycle >35 days at the operation were excluded.

As additional selection criteria, we balanced the samples according to the patient's cycle phase and included diverse endometriosis stages (rASRM I-IV), endometriosis types (DIE, endometrioma, peritoneal, or combination) and pain scores.

### Methodology

The biomarkers were identified through analysis of a patient cohort. Endometrial biopsies (Pipelles) from 42 patients were processed and stored under multiple conditions. The major part of the pipelle was digested into single-cell suspensions and cryopreserved for further analysis of gene expression. Any remaining sample has been stored in DMSO and/or flash frozen in liquid nitrogen to be used for the study of protein expression, isolation and culture of endometrial and immune cells present within the endometrial tissue.

Cell capture and cDNA library generation was performed using a Chromium system (10× Genomics). The cDNA library was sequenced using an Illumina platform.

### Quality of Sample

Only pipelle samples with single-cell viability >70% after thawing and >100′000 single-cells were included.

Pipelle quality observation: pipelles were assessed visually before being processed by single-cell dissociation.

### Patient Data

Medical data collected was curated into a format for integration into our internal deep learning platform ScaiVision™ or another suitable data analysis workflow that uses patient data as a tool to identify disease-related molecular profiles/or cell identity biomarkers.

### Data Analytics

### Data Pre-Processing

Quality control to check for technical or batch effects Automated cell-type annotation

Supervised discovery of predictive biomarkers using a convolutional neural network The goal is to identify and validate a gene signature that is predictive of one or more of the stated endpoints with a sensitivity and specificity ≥80%.

### Project Duration

24 months

### Statistical Analysis

We identified a set of biomarkers that define a signature present specifically in samples of endometriosis patients using the machine learning algorithm ScaiNet based on CellCnn (Arvaniti and Claasen 2017). To create an independent validation set, we randomly removed 40% of the 42 samples before starting network training. We considered any biomarker profile passing the accuracy significance threshold of >80% as a potential candidate. 3-fold cross-validation was used to evaluate the reproducibility of the ScaiNet algorithm on the specific dataset.

### Explanation of Work Done

Biomarker discovery was carried out on a cohort of endometrial samples (pipelles) collected from 256 patients directly prior to a planned surgery, 105 of which were subsequently diagnosed with endometriosis. Single-cell RNA sequencing was performed on 42 of the isolated pipelle samples measuring detectable levels of RNA transcripts in single cells in the endometrium tissue.

The workflow consists of steps for quality control of the raw read sequences, transcript quantification, quality control of the samples on a gene- and cell-level, normalisation, dimension reduction and sample class prediction using ScaiNet. The workflow is embedded in the workflow management engine Snakemake (Köster, J., & Rahmann, S. (2012). Bioinformatics, 28(19)) for automation and to ensure reproducibility.

Mapping and quantification of the raw reads are performed on the transcript level. Gene indexing is done by the Salmon package. Cell debarcoding, deduplication, read mapping, and estimation of transcript-level expression by pseudo-alignment using the Salmon alevin software. For Quality control (QC) of the raw reads, the software MultiQC (Ewels, P., Magnusson, M., Lundin, S., & Käller, M. (2016). Bioinformatics, 32(19)) is used. QC of the quantification step is done by the package AlevinQC (Charlotte Soneson and Avi Srivastava (2021). https://github.com/csoneson/alevinQC). The Seurat (Satija, R., Farrell, J. A., Gennert, D., Schier, A. F., & Regev, A. (2015) Nature Biotechnology, 33(5)) and scater (McCarthy, D. J., Campbell, K. R., Lun, A. T. L., & Wills, Q. F. (2017). Bioinformatics, 33(8)) packages are used to perform quality control and visualization of the data on the sample-, cell- and gene-level. Included is the detection and removal of outlier cells based on transcript and gene metrics, detection of possible doublet cells and batch effects.

Samples not fulfilling the following quality criteria were excluded; median cell-wise mitochondrial expression <30%, median number of genes per cell >1000, number of cells per sample >5000, median number of unique transcripts per cell >1000.

Dimension reduction is done by selecting highly variable genes that account for the most variation in a cell population. The selected features are then used to train ScaiNet for sample classification.

Patient samples were divided into two groups, consisting of those patients that were diagnosed with endometriosis during surgery and by pathology and non-endometriosis. This resulted in 24 endometriosis samples and 18 non-endometriosis samples.

Approximately 40% of samples from each group were set aside to use for model validation. The remaining 60% samples were used to train a series of ScaiNet neural networks to distinguish between the endometriosis and non-endometriosis groups. 50 to 100 such networks were trained with a range of chosen hyperparameters.

In order to generate a menstrual cycle phase independent model, we trained the network with samples from all patients (all menstrual phases; n=35). However, assuming that the cycle phase would influence the model, we separately trained a network on samples from patients in the proliferative phase (n=18, 9 non-endometriosis, 9 endometriosis).

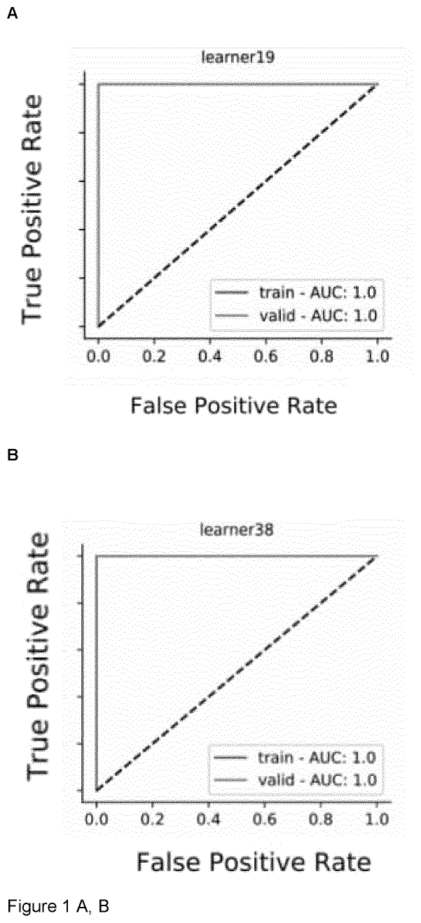

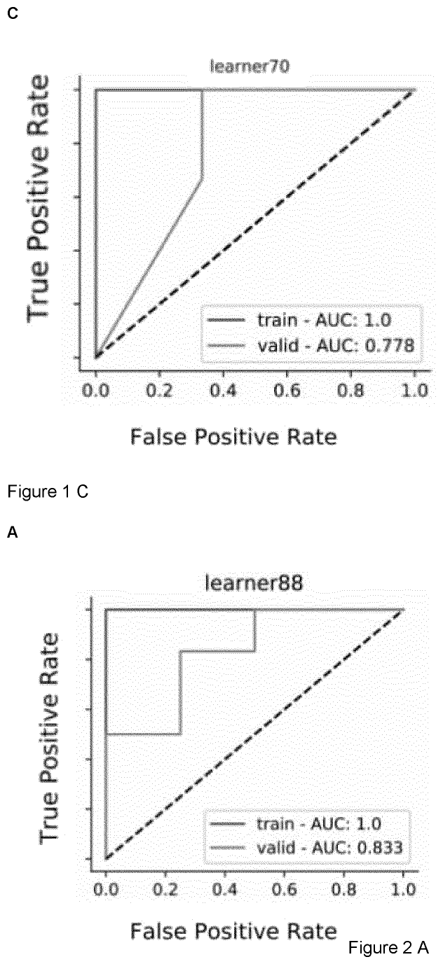

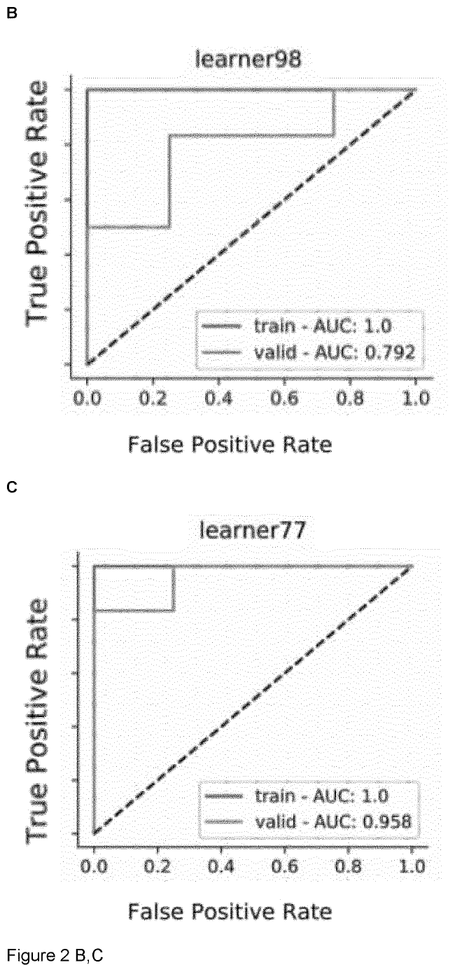

Three Cross-Validation (CV) splits with 60% of the samples were performed for training of ScaiNet. For proliferative samples, the best performing networks from the three independent CV splits achieving AUC of 1.00, 1.00, and 0.78, respectively, were selected for further analysis (FIG. 1). For all cycle samples, the best performing networks from the three independent CV splits achieving AUC of 0.83, 0.79, and 0.95, respectively, were selected for further analysis (FIG. 2).

In conclusion, the network efficiently identifies endometriosis patients, and with higher accuracies when the samples are collected during the proliferative phase than in any phases of the menstrual cycle.

### Example 2

ScaiNet learned filters from the best-performing networks, of which at least one was positively correlated with endometriosis and one negatively correlated with endometriosis (or positively correlated to non-endometriosis) (Table 1).

A gene signature was derived from the filters predicting endometriosis (Table 2 and 4) and non-endometriosis (Table 3 and 5) in the best-performing networks using the consensus from the top-weighted genes associated with endometriosis or non-endometriosis from all cycle phase samples or from proliferative samples, respectively.

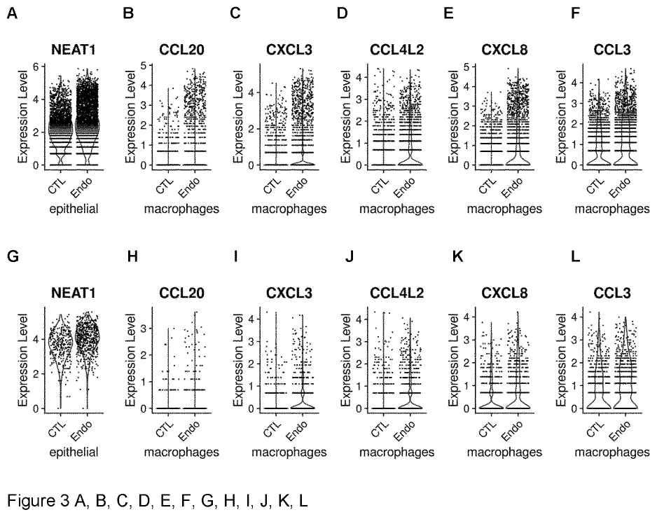

Each gene was identified by the weight assigned in the process of the model generation, which is used as an estimate of their influence on the prediction of endometriosis versus non-endometriosis. The gene signature was cross-validated through the differential expression analysis of all genes in the dataset. The analysis shows that an average 38% of the ScaiNet-derived predictive genes are differentially expressed between endometriosis versus non-endometriosis samples (Table 6 and 7). However, when separating proliferative samples from all cycle phase samples and performing a new differential expression analysis of all genes in the dataset, the overlap between differentially expressed genes and ScaiNet-derived predictive genes is 17% and 78%, respectively (FIGS. 3 A and B). The conspicuous overlap in this internal validation increases the confidence in ScaiNet predictions.

### Example 3

The discovered gene signatures were subjected to a gene ontology (GO) analysis to determine the categories of biological processes that are important or misregulated in the endometriosis samples. Among the top 50 GO terms and pathways ranked by the adjusted p-value are chemokine receptor activity and binding, and neutrophils and granulocytes chemotaxis and migration highlighting a role for the myeloid cell compartment of the immune system in endometriosis. Moreover, extracellular space and extracellular region are top ranked as cellular compartments (FIG. 4).

### Example 4

Using the weights from the filters positively or negatively correlated with endometriosis or non-endometriosis from the ScaiNet predictive networks, the inventors calculated filter response scores for every cell in the dataset, one per CV-split. These scores were used to determine the cells predictive of endometriosis in both proliferative and all cycle phase samples.

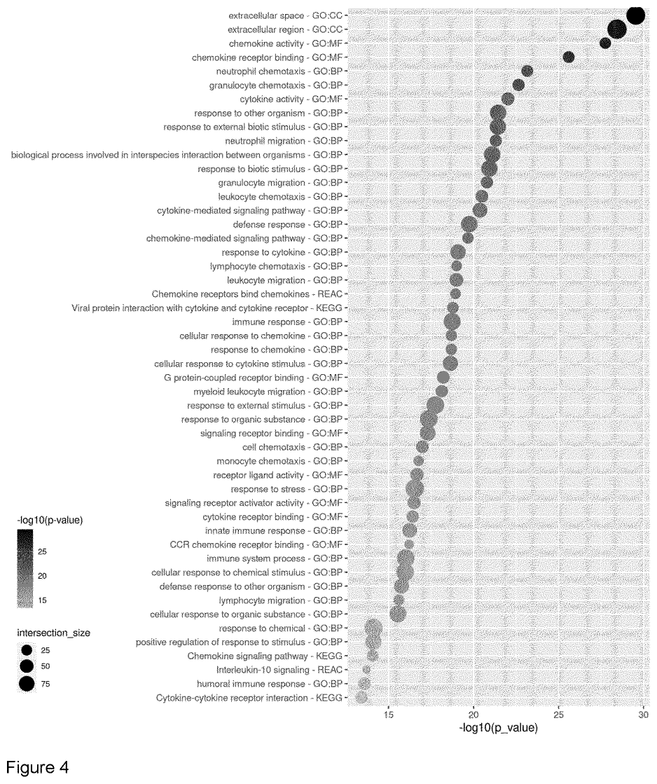

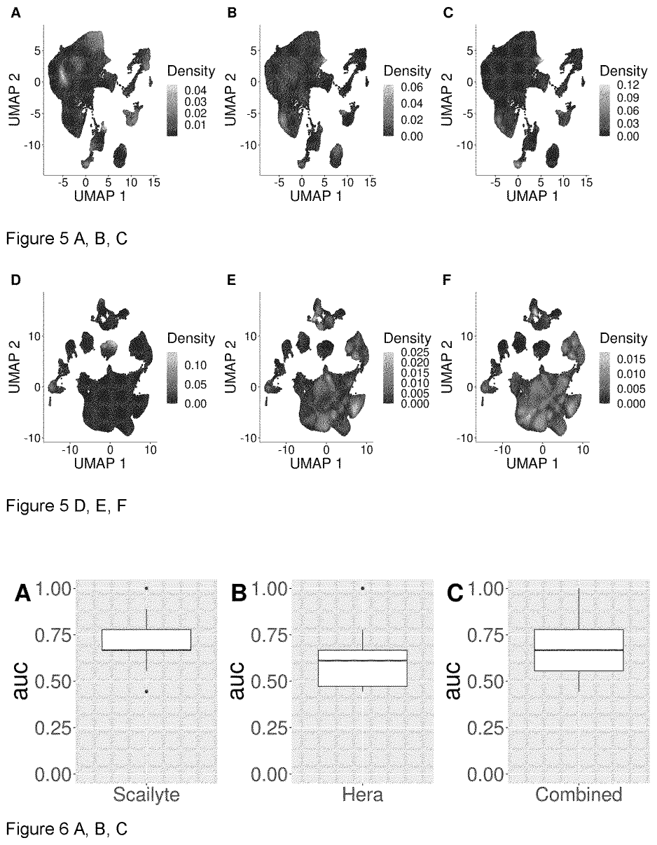

Interestingly, as determined/found by the GO analysis above, specific subsets of myeloid cells as well as epithelial cells (EC) and fibroblasts were identified to be expressing the biomarker gene signatures predictive for endometriosis in both proliferative and all cycle phase samples (FIGS. 5 A and B). The myeloid cells are characterized by following marker expression: CD14, CD16, CD15, CD11b The EC cells are defined by the expression of following genes: EpCAM and KRT18 The fibroblasts exhibited elevated expression of the following markers: COL18A1, COL4A2 COL4A1 VIM and CALD1.

TABLE 1
Biomarkers signature of endometriosis
Biomarkers signature of endometriosis

IGFBP1
IGFBP2
COL3A1
SFRP4
IFI44L
COL1A1
LY6E
PAEP
ISG15
TIMP1
IFI6
HSPA1A
DNAJB1
HSPA1B
RPL13P12
COL1A2
RPS2
MMP7
NEAT1
SPARC
RPL36A-HNRNPH2
XIST
LGALS1
RPS2P5
LDLR
MALAT1
ENSG00000260342 (Novel Protein)
ENSG00000248515 (Novel Transcript)
HMGCS1
LEFTY2
HLA-DQB1
HLA-DQA1
TIMP3
GNLY
IGFBP3
GPX3
HLA-DRB1
CXCL8
CXCL3
CCL20
IGKC
PHLDA1
CXCL5
PLIN2
CXCL2
CD163
NINJ1
TREM1
MGP
IL1RN
FTL
LUCAT1
DCN
EREG
MARCO
C3
CXCL1
CCL3
CCL4
CTSD
MMP19
TNF
CTSL
S100A9
LUM
HMOX1
LYVE1
CCL4L2
IL1B
HLA-DRB5
S100A8
IER3
NFKBIA
RNASE1
FN1
C15orf48
FTH1
DAB2
SOD2
CSTB
FBP1
PLAUR
IGFBP7
S100P
PCSK1N
LINC01320
HOMER2
NUPR1
MT1G
MT1F
CRIP1
CYP24A1
ITM2B
SLC7A2
SLC26A2
MT1X
ENSG00000285756 (Novel Transcript)
SPP1
AHNAK
FXYD2
SCCPDH
ANXA1
RBP1
LYPLAL1
STC1
APOL1
CTSC
MARCKSL1
IVNS1ABP
MT-ND4
RNASET2
HNMT
LAPTM4A
SLC40A1
ID1
ITGA2
UBC
SGK1
ALDH2
PROM1
SLC25A29
HMGCR
COMP
RXFP1
PAPSS1
MAFK
C2orf88
GRN
JUND
WFDC2
TUBB4B
ZNF90
FBLN1
IGF2
CMTM7
MUC1
POU5F1
MT-ND2
EZR
APOE
SPON1
NFKBIZ
SIK1B
CAPRIN1
ADIRF
ZFP36L1
SARAF
SLC3A1
PDK4
MT1E
MT1M
MT-ND3
TLE1
SOD3
MT1H
FOXO1
RIMKLB
BAG1
SCGB1D4
GADD45A
IRF1
PAX8
DEFB1
PPP1R15A
IGFBP4
SOX4
C19orf33
LBH
TNFAIP3
G0S2
APLP2
S100A6
GPR160
DUSP5
KLF5
DNAJC15
ENSG00000280138 (Novel Transcript)
EPHX1
GPX1
PIGR
GPRC5A
CYP3A5
USP53
SCGB1D2
WEE1
NDRG1
RAB11FIP1
TSPAN15
GEM
CRYAB
DSP
TNFSF10
SRD5A3
CLDN10
PDZK1IP1
EIF4A3
NNMT
LINC01502
ENSG00000280800 (Novel Transcript,
Similar To YY1 Associated Myogenesis
RNA 1 YAM1)
CITED2
CST4
S100A1
XACT
IL6ST
IFI27
SCGB2A1
PCK1
MT2A
RPS26
COTL1
CTSW
NKG7
KLRC1
FCER1G
GZMA
TYROBP
CAPG
PENK
YBX1
LMNA
EFEMP1
MZT2B
SPON2
RPL37
HLA-A
CEBPD
FABP5
HSPA5
IFITM1
IFITM3
CD81
PRSS23
THY1
YBX3
TUBA1A
IGFBP6
RPS29
NPC2
ISLR
CFD
GADD45B
CNN1
CALR
CEBPB
COL6A2
RPL36A
MYC
CXCL13
INAFM1
MYL9
VCAN
TGM2
PNRC1
BST2
ID4
SOCS3
DKK1
RASD1
FOSB
RPL23AP42
TAGLN
ZFP36
NPW
PTGDS
ACTA2
RPL7AP6
ERVMER34-1
TNFRSF18
FOXQ1
ENSG00000254732 (Novel Protein,
C11orf31-CTNND1 Readthrough)
MT-RNR2
CYBA
TMSB4X
LINC01480
MMP11
UGT2B7
IGF1
SAA1
TFF3
MT-ATP6
HSP90AA1
COL4A2
COL4A1
HAPLN1
MMRN1
ICAM1
SERPINE1
EGR1
HLA-B
FOS
DIO2
JUNB
BGN
IGHG1
IGKV3-20
IGKV1-39
IGLV2-14
H1-3
H4C3
TMSB10
SLC39A6
RHOB
FCGR2A
STAB1
GLUL
MKI67
HSPB1
SLPI
SERPINA1
MMP26
TNFAIP2
SCGB2A2
CCL19
LYZ
VWF
ENSG00000285417 (Novel
Pseudogene)
REN
NAPSB
C20orf85
CAPS
SAA2
A2M
CCL21
C1QB
JCHAIN
CD74
GZMB
XCL1
MZB1
MMP10
CCL18
CD14
C1QC
CST1
PPBP
MMP9
MMP1
HSPA6
TPPP3
RGS1
MS4A6A
WFDC21P
CCL3L1
TNFRSF11B
CDC20B
MMP3
SERPINB2
BCL2A1
CCL5
ENSG00000143248 (RGS5)
HOATZ
SAA2.SAA4
AGR3
AREG
S100A10
CXCL10
FCN1
ACTG2
ENSG00000258752 (Novel Transcript,
Antisense To FOXN3)
IGFBP5
CLDN3
CCL2
XCL2
IGHA1
IGLL5
CCL17
SNTN
AIF1
MS4A8
RSPH1
C9orf24
C1orf194
FAM183A
GPR183
CXCL14

TABLE 2
Biomarker signature associated to Endometriosis in all cycle phases
Biomarker signature associated to Endometriosis in all cycle phases

SCGB2A2
CCL19
LYZ
VWF
ENSG00000285417
REN
NAPSB
ENSG00000280800
C20orf85
WFDC2
CAPS
SAA2
A2M
CCL21
G0S2
PAEP
MT2A
CCL4
C1QB
MT1F
MARCO
JCHAIN
MT1G
GNLY
S100A8
CD74
SCGB2A1
HMOX1
GZMB
IL1B
XCL1
CTSW
MZB1
SPP1
MMP10

TABLE 3
Biomarker signature associated to non-Endometriosis in all cycle phases
Biomarker signature associated to non-Endometriosis in all cycle phases

IGFBP1
EREG
CCL18
GPX3
MARCO
JCHAIN
CD14
ENSG00000285417
IL1B
S100A8
SAA1
CXCL3
MMP7
MT2A
RIMKLB
LEFTY2
TIMP3
G0S2
NKG7
ENSG00000280800
SCGB1D4
C1QC
MT1G
CCL4L2
GZMA
CST1
CCL3
CTSW
XCL1
REN
PPBP
WFDC2
SLPI
CXCL5
CCL4
SCGB1D2
TYROBP
CCL20
NEAT1
CXCL8
SAA2
S100P
DEFB1
MMP9
MZB1
MMP1

TABLE 4
Biomarker signature associated to Endometriosis in proliferative phase
Biomarker signature associated to Endometriosis in proliferative phase

PENK
NEAT1
PAEP
HSPA6
TPPP3
MMP9
TFF3
HMOX1
RGS1
MS4A6A
LYZ
SPP1
WFDC21P
IGFBP3
CCL3L1
TNFRSF11B
IL1RN
CDC20B
MMP3
SERPINB2
MMP1
REN
BCL2A1
GZMB
CCL5
ENSG00000143248
ENSG00000285417
HOATZ
SAA2.SAA4
AGR3
AREG
RNASE1
S100A10
SAA2
CXCL10
CRIP1
VWF
STC1
FCN1
TAGLN
ACTG2
ENSG00000258752
IGFBP5
CLDN3
CCL2
LINC01480
MT2A

TABLE 5
Biomarker signature associated to non-Endometriosis in proliferative phase
Biomarker signature associated to non-Endometriosis in proliferative phase

IGFBP5
MALAT1
MARCO
CLDN3
CCL18
RNASE1
CD14
S100A10
XCL2
CXCL3
AREG
IGHA1
EREG
IGLL5
ACTG2
SAA2
MMP7
SAA2.SAA4
CCL20
CCL17
MKI67
SNTN
HOATZ
AGR3
AIF1
MS4A8
RSPH1
LYZ
C9orf24
C1orf194
FAM183A
SLPI
GPR183
TIMP1
TPPP3
C20orf85
CCL4L2
PAEP
CXCL14
CAPS
ENSG00000280800
PENK

TABLE 6
Differentially expressed gene signature in Endometriosis in all phases
Differentially expressed gene signature in Endometriosis in all phases

IGFBP1
IGFBP2
COL3A1
SFRP4
IFI44L
COL1A1
LY6E
PAEP
ISG15
TIMP1
IFI6
HSPA1A
DNAJB1
HSPA1B
RPL13P12
COL1A2
RPS2
MMP7
NEAT1
SPARC
RPL36A-HNRNPH2
XIST
LGALS1
RPS2P5
LDLR
MALAT1
ENSG00000260342
ENSG00000248515
HMGCS1
LEFTY2
HLA-DQB1
HLA-DQA1
TIMP3
GNLY
IGFBP3
GPX3
HLA-DRB1
CXCL8
CXCL3
CCL20
IGKC
PHLDA1
CXCL5
PLIN2
CXCL2
CD163
NINJ1
TREM1
MGP
IL1RN
FTL
LUCAT1
DCN
EREG
MARCO
C3
CXCL1
CCL3
CCL4
CTSD
MMP19
TNF
CTSL
S100A9
LUM
HMOX1
LYVE1
CCL4L2
IL1B
HLA-DRB5
S100A8
IER3
NFKBIA
RNASE1
FN1
C15orf48
FTH1
DAB2
SOD2
CSTB
FBP1
PLAUR
IGFBP7
S100P
PCSK1N
LINC01320
HOMER2
NUPR1
MT1G
MT1F
CRIP1
CYP24A1
ITM2B
SLC7A2
SLC26A2
MT1X
ENSG00000285756
SPP1
AHNAK
FXYD2
SCCPDH
ANXA1
RBP1
LYPLAL1
STC1
APOL1
CTSC
MARCKSL1
IVNS1ABP
MT-ND4
RNASET2
HNMT
LAPTM4A
SLC40A1
ID1
ITGA2
UBC
SGK1
ALDH2
PROM1
SLC25A29
HMGCR
COMP
RXFP1
PAPSS1
MAFK
C2orf88
GRN
JUND
WFDC2
TUBB4B
ZNF90
FBLN1
IGF2
CMTM7
MUC1
POU5F1
MT-ND2
EZR
APOE
SPON1
NFKBIZ
SIK1B
CAPRIN1
ADIRF
ZFP36L1
SARAF
SLC3A1
PDK4
MT1E
MT1M
MT-ND3
TLE1
SOD3
MT1H
FOXO1
RIMKLB
BAG1
SCGB1D4
GADD45A
IRF1
PAX8
DEFB1
PPP1R15A
IGFBP4
SOX4
C19orf33
LBH
TNFAIP3
G0S2
APLP2
S100A6
GPR160
DUSP5
KLF5
DNAJC15
ENSG00000280138
EPHX1
GPX1
PIGR
GPRC5A
CYP3A5
USP53
SCGB1D2
WEE1
NDRG1
RAB11FIP1
TSPAN15
GEM
CRYAB
DSP
TNFSF10
SRD5A3
CLDN10
PDZK1IP1
EIF4A3
NNMT
LINC01502
ENSG00000280800
CITED2
CST4
S100A1
XACT
IL6ST
IFI27
SCGB2A1
PCK1
MT2A
RPS26
COTL1
CTSW
NKG7
KLRC1
FCER1G
GZMA
TYROBP
CAPG
PENK
YBX1
LMNA
EFEMP1
MZT2B
SPON2
RPL37
HLA-A
CEBPD
FABP5
HSPA5
IFITM1
IFITM3
CD81
PRSS23
THY1
YBX3
TUBA1A
IGFBP6
RPS29
NPC2
ISLR
CFD
GADD45B
CNN1
CALR
CEBPB
COL6A2
RPL36A
MYC
CXCL13
INAFM1
MYL9
VCAN
TGM2
PNRC1
BST2
ID4
SOCS3
DKK 1.00
RASD1
FOSB
RPL23AP42
TAGLN
ZFP36
NPW
PTGDS
ACTA2
RPL7AP6

TABLE 7
Differentially expressed gene signature in Endometriosis in
proliferative phase
Differentially expressed gene signature in Endometriosis in
proliferative phase

RPL13P12
ERVMER34-1
TNFRSF18
FOXQ1
ENSG00000254732
MT-RNR2
LINC01320
COL3A1
COL1A2
CYBA
TMSB4X
COL1A1
LGALS1
LINC01480
CEBPD
PNRC1
MMP11
SFRP4
UGT2B7
IGF1
SPARC
SAA1
TFF3
HLA-DRB5
MT-ATP6
RPS2
HLA-DRB1
HSP90AA1
COL4A2
FN1
COL4A1
HAPLN1
NEAT1
DNAJB1
HSPA1B
HSPA1A
MMRN1
ICAM1
SERPINE1
IGKC
EGR1
HLA-A
HLA-B
PENK
IGF2
MGP
LUM
DCN
FOS
DIO2
JUNB
ZFP36
BGN
IGHG1
IGKV3-20
IGKV1-39
IGLV2-14
IGFBP3
H1-3
H4C3
ITM2B
TMSB10
SLC39A6
RHOB
TAGLN
ACTA2
HLA-DQB1
HLA-DQA1
FCGR2A
STAB1
GLUL
S100A9
CTSD
CXCL3
MKI67
SOD2
CXCL8
CTSL
RPS2P5
IFITM1
SOD3
ENSG00000280800
DEFB1
HSPB1
SLPI
SERPINA1
MMP26
RPS26
PAEP
TNFAIP2

### Example 5

Comparison of the predictive value of our signature in the diagnosis of endometriosis with a previously published gene signature by candidate selection (Chen-Wei Chen, et al., 2021 bioRxiv 2021.01.25.428135) highlights the superiority of our method in the context of endometriosis detection as well as of the inventors' unbiased approach (FIG. 6). Indeed, as shown by the average median AUC score of 0.78 for the inventors' model, the competing signature performed poorly on our dataset (average median AUC=0.56) and its combination with the inventors' model did not add any value to the prediction score (average median AUC=0.78) (average median AUC of three independent CV-splits).

### Example 6

### Planned Validation

An independent validation cohort of 30-40 patients will be recruited, consisting of 50% endometriosis and 50% non-endometriosis patients. Endometrium biopsies will be obtained from the patients in the validation cohort during different cycle phases and will be analyzed using the same single-cell RNA-sequencing method as for the discovery cohort. The same pre-processing steps for the data will be applied.

The endometriosis probability for each patient of the validation cohort will be predicted using the best-performing ScaiNet network trained on the discovery cohort. Using single-cell RNA-sequencing data alone, we will aim at achieving accuracies of AUC >0.85.

By integrating clinical data into the ScaiNet networks we aim at further improving accuracy of the prediction

The endometriosis probability for the patients in the validation cohort will be determined using an optimized and reduced set of genes from Table 1. Characterization of each gene and its performance will be thoroughly assessed in several human tissue types to provide a comprehensive, specific and sensitive assay for the diagnosis of endometriosis, and to infer or exclude the diagnosis of other diseases or disorders of the female reproductive tract (e.g. ovarian and endometrial cancer).

Using nucleotide measurements (e.g. RNA) and protein measurements (e.g. FACS and ELISA), we will extend and specialize the use of our panel. Comparisons with other methods used in diseases or disorders of the female reproductive tract will be performed.

Finally, our invention will be benchmarked against the gold standard method for diagnosis and treatment of diseases or disorders of the female reproductive tract (i.e. surgical laparoscopy for endometriosis).

### Example 7

The inventors identified biomarkers that are differentially expressed during the proliferative phase compared to the secretory phase of the menstrual cycle. These markers can be used to identify the menstrual cycle phase in silico, in particular the proliferative phase solely based on the RNA in the sample or based on the RNA in the sample in combination with other data such as body temperature measurements, secret viscosity measurements and/or patient background data such as days since the last menstruation or past cycle length.

TABLE 8
Menstrual cycle markers
Differentially expressed gene signature in menstrual cycle phase

PLA2G4F
GAST
TMEM61
CYP26A1
GJB1
SULT1E1
LHFPL3
PLEKHF2
A2ML1
TBC1D3E
LOC101927020
C5AR2
ENSG00000276122
DIPK1C
MEDAG
CPEB2
LTBP2
ALDH1A3
RXFP1
SLC25A48
ENSG00000260186
STEAP4
PDZD2
RYR3
ENSG00000237949
MT1H
DMRTA1
LINC01010
MRPS6
NPR3
HGD
SMIM24
ENSG00000278898
ZBTB16
GCNT3
ANKRD35
KCNN4
PLLP
TLR5
GALNT5
FKBP5
PDE6A
FEZF1-AS1
WIPI1
CCN3
MT1G
AUP1
KCNK13
XDH
ENSG00000229385
HSD17B2
GNPDA1
ACP3
TM4SF4
HPCAL4
PCDH1
MMP11
CD36
PITPNM3
FAM110C
LHFPL3-AS1
HEXIM1
LLGL2
DUOXA1
SFTA2
BIRC3
PPM1B
ATP1A1
POPDC3
JADE1
SEMA3D
CXCL13
ENSG00000262061
MT1M
TCN1
ENSG00000218672
DISC1FP1
ITGB8
IVNS1ABP
CNDP2
TOX2
SLC30A2
LINC01133
FLVCR2-AS1
CARD10
FMO5
ENSG00000205424
TM4SF1
PPP2R2C
CLDN8
ENSG00000284309
S100P
DUOX1
SYT1
BNIP3
PRG4
PLCL1
SLC3A1
LRRC1
PRKX

1. A method for determining an endometriosis state in an endometrial tissue sample of a female subject, the method comprising the steps of:
i) determining RNA levels of at least two biomarkers in an endometrial tissue sample of a female subject, wherein the biomarkers comprise or consist of:
a) CCL5 and/or NEAT1; and/or
b) further biomarker(s) selected from Table 1; and
ii) determining an endometriosis status in the endometrial tissue sample based on the RNA levels of the at least two biomarkers of i).

2. The method of claim 1, wherein
a) i) an alteration compared to a reference value of one or more biomarkers selected from Table 2 is indicative of an endometriosis disease state; and/or
ii) an alteration compared to a reference value of NEAT1 and/or (a) further biomarker(s) selected from Table 3 is indicative of an non-endometriosis disease state; and
b) wherein the reference value is indicative of a healthy status.

3. A method for determining an endometriosis state based on a plurality of endometrial cells, the method comprising the steps of:
i) determining a frequency of cells expressing RNA of at least two biomarkers in a plurality of cells of an endometrial sample of a female subject, wherein the biomarkers comprise or consist of:
a) CCL5 and/or NEAT1; and/or
b) further biomarker(s) selected from Table 1; and
ii) determining an endometriosis state based on the frequency determined in i).

4. The method of claim 3, wherein
a) i) an altered frequency of cells expressing one or more biomarkers selected from Table 2 compared to a reference frequency is indicative of an endometriosis disease state; and/or
ii) an altered frequency of cells expressing of NEAT1 and/or (a) further biomarker(s) selected from Table 3 compared to a reference frequency is indicative of a non-endometriosis disease state; and
b) wherein the reference value is indicative of a healthy status.

5. The method of any one of claims 1 to 4, wherein the method comprises at least one step of pre-selecting cells
i) selected from the group consisting of:
a) immune cells selected from the group consisting of B-cells, T-cells, dendritic cells and macrophages;
b) epithelial cells selected from the group consisting of basal cells, ciliated cells and unciliated cells;
c) endothelial cells; and
d) smooth muscle cells; and/or
ii) with at least one cell lineage marker, preferably using at least one cell lineage marker selected from the group of:
a) CD14, CD16, CD45, CD15, CD11b;
b) EpCAM and KRT18; and
c) COL18A1, COL4A2, COL4A1, VIM or CALD1.

6. The method of any one of claims 1 to 5, wherein at least 3, 4, 5, 6, 7, 8 or 9 biomarkers are determined.

7. The method of any one of claims 1 to 6, wherein the method additionally comprises determining or retrieving at least one non-molecular marker, preferably wherein the non-molecular marker comprises a marker selected from the group consisting of: age, weight, BMI, gravidity, parity, ethnicity, fertility status, previous laparoscopies, previous use of medication and other gynaecological disorders.

8. The method of any one of claims 1 to 7, wherein the method is at least partially computer-implemented and wherein the RNA levels are determined by retrieving data indicative for the RNA levels.

9. The method of any one of claims 1 to 8, wherein the sample is a proliferative phase sample or wherein the cells are cells obtained during the proliferative phase.

10. The method of claim 9, wherein an increase of one or more biomarkers selected from Table 4 is indicative of an endometriosis disease state and/or wherein an increase of one or more biomarkers selected from Table 5 is indicative of non-endometriosis disease state.

11. A method for determining the validity of an endometriosis state, the method comprising the steps of:
i) determining an endometriosis state according to claim 1 to 8;
ii) determining or retrieving the menstrual cycle status of the subject at the timepoint of obtainment of the endometrial cells or of the endometrial sample;
iii) determining the validity of the endometriosis state, based on the menstrual cycle status, preferably wherein the validity is considered higher if the menstrual cycle status is the proliferative phase than if the menstrual cycle status is in a different menstrual cycle state.

12. The method of claim 11, wherein determining the menstrual cycle status comprises determining the RNA level of at least one menstrual cycle biomarker.

13. The method of claim 12, wherein the menstrual cycle marker comprises a marker from Table 8.

14. A method for prediction of disease outcome, disease development and/or disease progression of a female subject having endometriosis or at risk of having a endometriosis, the method comprising the steps of:
a) determining an endometriosis state according to the method of any one of claims 1 to 10;
b) comparing the endometriosis state determined in a) to a prediction reference pattern; and
c) predicting disease outcome disease development and/or disease progression of the female subject based on the comparison in step b).

15. The method of claim 13, wherein
1.) determining an increased frequency of cells expressing the biomarker(s) from Table 2 compared to the prediction reference pattern; and/or
2.) determining an increased level of the biomarker(s) from Table 2 compared to the reference pattern
is indicative for worsening of disease outcome more likely disease development and/or more likely disease progression.

16. The method of claim 14 or 15, wherein
1.) determining an increased frequency of cells expressing the biomarker(s) from Table 3 compared to the prediction reference pattern; and/or
2.) determining an increased level of NEAT and/or (a) further biomarker(s) from Table 3 compared to the reference pattern
is indicative for improvement of disease outcome less likely disease development and/or less likely disease progression.

17. A method for predicting susceptibility to a treatment for endometriosis of a female subject having endometriosis or at risk of having endometriosis, the method comprising the steps of:
a) a) determining an endometriosis state according to the method of any one of claims 1 to 10;
b) comparing the endometriosis state to a susceptibility reference pattern; and
c) predicting susceptibility to a treatment for endometriosis of the female subject based on the comparison in step b).

18. The method of claim 17 wherein the treatment for endometriosis is a treatment selected from the group consisting of: pain medication, hormonal therapy, fertility treatment and surgery.

19. The method of any one of claims 14 to 18, wherein the susceptibility reference pattern or the prediction reference pattern is obtained from reference subjects, wherein at least one of the reference subjects has been diagnosed with endometriosis.

20. The method of claim 19, wherein obtaining the susceptibility reference pattern or the prediction reference pattern from reference subjects comprises a machine-learning technique, preferably a convolutional neural network and/or logistic regression.

21. A method for classification of a female subject having a endometriosis or at risk of having a endometriosis into a class, the method comprising the steps of:
b. i) determining an endometriosis state according to the method of any one of claims 1 to 10;
ii) predicting a disease outcome, disease development and/or disease progression of a female subject according to the method of any one of claims 14 to 16, 19 or 20; and/or
iii) predicting susceptibility to a treatment for endometriosis of a female subject according to the method of any one of claims 17 to 20; and
c. classifying the female subject according to the frequency determined in i), signature determined in ii), prediction of iii), and/or prediction in iv).

22. The method of claim 21, wherein at least one class is indicative of the stage and/or severity of the endometriosis.

23. A composition comprising reagents for the detection of biomarkers for the diagnosis of endometriosis, the biomarkers comprising or consisting of at least two markers from Table 1.

24. A pharmaceutical product comprising a compound against endometriosis for use in treatment of a female subject that is predicted as susceptible to a treatment for endometriosis according to the method of claim 17 to 20.

25. The pharmaceutical product of claim 24, wherein the compound against endometriosis is selected from the group of: ibuprofen, naproxen, oxycodone, desogestrel, dienogest, levonorgestrel, clomiphene citrate, gonadotropins, metformin, letrozole and bromocriptine.

26. The method of any one of claims 1 to 22, the composition of claim 23 or the pharmaceutical product of claim 24 or 25, wherein the endometriosis is selected from the group of: peritoneal endometriosis, endometriomas, deeply infiltrating endometriosis, tubal endometriosis and abdominal wall endometriosis.

27. The method of any one of claims 1 to 22, 26, the composition of claim 23 or 26 or the pharmaceutical product of any one of claims 24, 25 or 26, wherein the endometriosis at rASRM II, III, or IV stage.

28. A computer program product comprising instructions to execute the method of any one of claims 8 to 22, 26, 27, wherein the method is computer-implemented.
